(19) United States
(12) Patent Application Publication
(10) Pub. No.: US 2018/0318263 A1
(43) Pub. Date: Nov. 8, 2018

(54) TREATMENT OF PROSTATE CANCER CELLS WITH FAT OXIDATION INHIBITORS AND ENZALUTAMIDE

(71) Applicant: The Regents of the University of Colorado, a body corporate, Denver, CO (US)

(72) Inventor: Isabel Rubio Schlaepfer, Aurora, CO (US)

(21) Appl. No.: 16/026,758

(22) Filed: Jul. 3, 2018

Related U.S. Application Data
(63) Continuation of application No. PCT/US2017/012167, filed on Jan. 4, 2017.
(60) Provisional application No. 62/274,564, filed on Jan. 4, 2016.

(51) Int. Cl. A61K 31/4166; A61P 35/00; A61K 31/495; A61K 31/336; A61K 31/415; A61K 31/58; A61K 31/277; A61K 31/4439; A61K 31/4458

(52) U.S. Cl. CPC A61K 31/4166; A61P 35/00; A61K 31/495; A61K 31/336; A61K 31/4458; A61K 31/58; A61K 31/277; A61K 31/4439; A61K 31/415

(57) ABSTRACT

A combination therapy for the treatment of cancers refractory to anti-androgen therapy; particularly prostate cancer. The combination therapy includes a pharmaceutically effective amount of enzalutamide, bicalutamide, and/or abiraterone combination with ranolazine, perhexiline, and/or etomoxir. The invention also includes methods for treating cancer. The method can include the steps of administering to the subject a pharmaceutically effective amount of an anti-androgen drug in combination with a pharmaceutically effective amount of a drug that block or reduces lipid metabolism. The cancer can be prostate cancer. In a beneficial embodiment the prostate cancer is unresponsive or has a reduced responsiveness to anti-androgen treatment. The pharmaceutically effective amount of an anti-androgen drug can enzalutamide and abiraterone. It can also be combinations, analogs and derivatives of enzalutamide and/or abiraterone. The pharmaceutically effective amount of a drug that blocks or reduces lipid metabolism can be ranolazine, perhexiline, or etomoxir. It can also be combinations, analogs and derivatives of ranolazine, perhexiline, and/or etomoxir.

### CROSS-REFERENCE TO RELATED APPLICATIONS

This application is a continuation of International Patent Application No. PCT/US2017/012167, filed Jan. 4, 2017, which claims the benefit of U.S. Provisional Patent Application No. 62/274,564, filed Jan. 4, 2016.

### STATEMENT OF GOVERNMENT INTEREST

This invention was made with Government support under Grant No. CA168934 awarded by the National Institutes of Health. The Government has certain rights in the invention.

### FIELD OF INVENTION

This invention relates to the treatment of cancer. More specifically, this invention relates to the treatment of prostate cancer with a combination of a fat oxidation inhibitor and an anti-androgen drug, such as enzalutamide.

### BACKGROUND OF THE INVENTION

Prostate cancer (PCa) is the most commonly diagnosed malignancy and the second highest contributor to cancer deaths in men in the United States (Carlsson, S, et al., 2012. J. Clin. Oncol. 30:2581-2584). Currently, the standard systemic treatment for advanced PCa is based on androgen deprivation with initial positive responses, but PCa tumors eventually become resistant and restore androgen receptor (AR) signaling (Knudsen, K. E., and Scher, H. I. 2009. Clin. Cancer Res. 15:4792-4798). After PCa becomes castration-resistant no curative treatments exist, making the identification of novel therapies imperative. The way castration-resistant tumors activate lipid metabolism is unknown, but likely involves a gene expression program orchestrated by novel and restored AR signaling pathways (Dehm, S. M., and Tindall, D. J. 2011. Endocr. Relat Cancer 18: R183-R196).

### SUMMARY OF THE INVENTION

The long-standing but heretofore unfulfilled need for is now met by a new, useful, and nonobvious invention.

In a first aspect the present invention provides a method of treating cancer in a subject. The method includes the steps of administering to the subject a pharmaceutically effective amount of an anti-androgen drug in combination with a pharmaceutically effective amount of a drug that blocks or reduces lipid metabolism. The cancer can be prostate cancer. In certain embodiments the prostate cancer is unresponsive or has a reduced responsiveness to anti-androgen treatment. In other embodiments the pharmaceutically effective amount of a drug that blocks or reduces lipid metabolism enhances the effect of an anti-androgen drug. The pharmaceutically effective amount of an anti-androgen drug can be enzalutamide, bicalutamide, abiraterone, galeterone, and/or AR-509. It can also be combinations, analogs and derivatives of enzalutamide, bicalutamide, abiraterone, galeterone, and/or AR-509. The pharmaceutically effective amount of a drug that blocks or reduces lipid metabolism can be ranolazine, perhexiline, or etomoxir. It can also be combinations, analogs and derivatives of ranolazine, perhexiline, and/or etomoxir.

In a second aspect the present invention provides a second method of treating cancer in a subject. The method includes the steps of administering to the subject a pharmaceutically effective amount of an anti-androgen drug assessing the responsiveness of the cancer to the administration of the anti-androgen drug, and administering to the subject a pharmaceutically effective amount of a drug that blocks or reduces lipid metabolism responsiveness to the assessment that the cancer of the subject has limited or no response to the anti-androgen drug. The cancer can be prostate cancer. The pharmaceutically effective amount of an anti-androgen drug can be enzalutamide, bicalutamide, abiraterone, galeterone, and/or AR-509. It can also be combinations, analogs and derivatives of enzalutamide, bicalutamide, abiraterone, galeterone, and/or AR-509. The pharmaceutically effective amount of a drug that block or reduces lipid metabolism can be ranolazine, perhexiline, or etomoxir. It can also be combinations, analogs and derivatives of ranolazine, perhexiline, and/or etomoxir.

In a third aspect the present invention provides a third method of treating cancer in a subject. The method includes the steps of pretreating the subject with a pharmaceutically effective amount of an anti-androgen drug and treating the subject with a pharmaceutically effective amount of a drug that blocks or reduces lipid metabolism. The treatment with the pharmaceutically effective amount of a drug that blocks or reduces lipid metabolism can be performed in response to an assessment that the cancer of the subject has limited or no response to the anti-androgen drug. The cancer can be prostate cancer. The pharmaceutically effective amount of an anti-androgen drug can be enzalutamide, bicalutamide, abiraterone, galeterone, and/or AR-509. It can also be combinations, analogs and derivatives of enzalutamide, bicalutamide, abiraterone, galeterone, and/or AR-509. The pharmaceutically effective amount of a drug that blocks or reduces lipid metabolism can be ranolazine, perhexiline, or etomoxir. It can also be combinations, analogs and derivatives of ranolazine, perhexiline, and/or etomoxir.

In a fourth aspect the present invention provides a fourth method of treating cancer in a subject. The method includes the steps of administering to the subject a pharmaceutically effective amount of a drug selected from the group consisting of ranolazine, perhexiline, etomoxir and combinations, analogs and derivatives thereof responsive to the detection of enzalutamide-resistant prostate cancer in the subject. The method can further include the step of administering celecoxib to the subject.

In a fifth aspect the present invention provides a combination therapy for the treatment of cancers refractory to anti-androgen therapy. The combination therapy can include a pharmaceutically effective amount of an anti-androgen drug selected from the group consisting of enzalutamide, bicalutamide, abiraterone, galeterone, and/or AR-509, and combinations, analogs and derivatives thereof; and a pharmaceutically effective amount of a drug that block or reduces lipid metabolism selected from the group consisting of ranolazine, perhexiline, etomoxir, and combinations, analogs and derivatives thereof.

In a sixth aspect the present invention provides a method for reversing prostate cancer cell resistance to anti-androgen drugs. The method can include the step of contacting prostate cancer cells with an effective amount of a drug that block or reduces lipid metabolism. In certain embodiments the drug that blocks or reduces lipid metabolism is ranolazine, perhexiline, and/or etomoxir. The contacted prostate cancer cells can be in a subject and the subject can be in need of a treatment for prostate cancer.

### BRIEF DESCRIPTION OF THE DRAWINGS

For a fuller understanding of the invention, reference should be made to the following detailed description, taken in connection with the accompanying drawings, in which:

FIG. 1 is a diagram of the site of action of the metabolic drugs to be used in the combination: Etomoxir and Perhexiline inhibit entry of fatty acids into mitochondria via CPT1. Ranolazine decreases beta-oxidation. Lipid synthesis mediators like ACC and FASN are also indicated. Fatty acids from lipid droplets (TG=triglyceride storage, SCD1=Steroyl-CoA-Desaturase) or from the media can be made available to mitochondria for β-oxidation.

FIG. 2 is a set of four graphs (FIGS. 2A-2D), three images (FIGS. 2E-2F), and a diagram (FIG. 2G) showing that Etomoxir decreases the viability of PCa cells in vivo and in vitro. Etomoxir (A) and Ranolazine (B) dose-curves in LNCaP cells. (C) Decreased VCaP tumor growth in mice treated with etomoxir for 21 days *p<0.05. (D) Effect of etomoxir (75 μM) on viability in benign (BPH-1 and WPMY-1) and PCa cell lines, *p<0.01. (E) Stain of xenografts slices with phospho-4EBP1 antibody. (F) Western blot of LNCaP cells treated with vehicle (“V”) or etomoxir (“E”), C-Casp3=cleaved-caspase 3. (G) Putative diagram of mTOR signaling pathway.

FIG. 3 is a pair of images showing CPT1A stain of human prostate cancer: (A) normal glandular tissue, 40×. Brown stain is only located in the epithelium. (B) Stain of a Gleason 5 Pattern cancer specimen, 40×. Nucleus is stained in blue. CPT1A is brown and has stronger signal than in normal tissue.

FIG. 4 is a pair of graphs (FIGS. 4A and 4B) and a set of images (FIGS. 4C and 4D) showing increased ER stress markers in etomoxir-treated LNCaP cells: Gene expression of ATF4, GADD34 and the GRP78 chaperone (A), and CCAAT-homologous protein or CHOP (B), *p<0.001. (C) Phosphorylation of the α-subunit of the translation initiation factor eIF2 (p-eIF2a) and expression of LC3 cleavage marker of autophagy. (D) Agarose gel showing the spliced (activated, 447 bp) XBP1 factor that regulates the response to ER stress. Unresolved ER stress leads to ceramide accumulation and apoptosis. (ATF4—left; GADD34—center; and GRP78—right for each replicate in FIG. 4A.)

FIG. 5 is a pair of graphs (FIGS. 5A and 5C) and an image (FIG. 5B) showing that lipid oxidation blockade decreases AR content and action and synergizes with enzalutamide to decrease LNCaP growth. (A) Relative AR gene expression after vehicle (“V”) or etomoxir (“E”) treatment in LNCaP cells. ARfl=full length AR; ARv7=AR variant 7; AR total=all AR isoforms; PSA=prostate specific antigen; NKX3.1=Homeodomain-containing transcription factor regulated by androgens. Similar results were obtained with androgen-independent VCAP cells (data not shown). All genes were significant from vehicle: p<0.05. (B) Western blot of full length AR and ARV7 variant in LNCaP cells treated with etomoxir for 48 hours. The AR-v7 bands were obtained by IP since this variant is not abundant in LNCaP cells. (C) MTS proliferation assay of LNCaP cells exposed to increasing concentrations of enzalutamide (μM) and etomoxir (μM) for 48 hours.*p<0.01 compared to single drug treatments.

FIG. 6 is a set of three graphs showing increased AR gene expression and sensitivity to enzalutamide in LNCaP CPT1A-KD clones: Expression of AR-full length (A) and ARv7 variant (B) in LNCaP-KD clones treated with enzalutamide (Enza) over 2 days. (C) Relative growth of LNCaP-KD clones compared to control (NTshRNA) with Enza treatment. *p<0.001, compared to control treatment. ap<0.01 compared to control clone. (NTshRNA—left; CPT1Ash1—center; and CPT1 Ash2 at each timepoint for FIGS. 6A and 6B.)

FIG. 7 is a pair of graphs showing the assessment of enzalutamide sensitivity in VCaP (A) and LNCaP (B) cells. Parental (lighter bars on the left) and enzalutamide resistant cells (darker bars on the right) were grown in the presence of increasing concentrations of enzalutamide (MDV3100). Colony formation was examined after 2 weeks by crystal violet stain and analyzed with image J.

FIG. 8 is a pair of graphs and an image of a Western blot showing that Ranolazine inhibits growth of LNCaP and VCaP-Enza-Resistant cells and decreases AR expression. Ranolazine dose-response in LNCaP parental and enzalutamide-resistant cells (A) and VCaP parental and enzalutamide-resistant cells (B) treated for 48 hours. (C) Western blot of VCaP cells treated with vehicle (“V”), Etomoxir (“E”) or Ranolazine (“R”) for 48 hours. The enzalutamide resistant (“Enza-res”) lysates are in the left three lanes of the Western blot (FIG. 8C).

FIG. 9 is an image showing CPT1A genomic editing with CRISPR technology. CPT1A expression in isolated clones. C1 and C2 are non-targeting CRISPR controls, p=parental LNCaP cells.

FIG. 10 is an image and a graph showing mouse TRAMPC1 cell growth assay. The graph shows relative growth of cells after treatments, *p<0.01 compared to vehicle. Representative crystal violet stains are shown above graph. RANO=ranolazine (100 M), MDV=enzalutamide (5 μM).

FIG. 11 is a pair of diagrams showing that CPT1-mediated lipid oxidation supports PCa growth. (A) Fatty acids are oxidized in mitochondria via CPT1 translocation, sustaining growth and endoplasmic reticulum (“ER”) homeostasis. Androgen action supports these pathways inducing lipogenesis and beta-oxidation. (B) CPT1 blockade results in decreased growth, compensatory increase in AR and accumulation of fatty acids leading to ceramide production and ER stress, ultimately leading to apoptosis. The transient compensatory increase in AR to increase beta-oxidation further aggravates the lipotoxicity and makes these metabolically-challenged cells more sensitive to anti-androgen therapy. CPT1A is a liver isoform abundant in PCa cells.

FIG. 12 is a pair of images and associated tables illustrating that mouse prostate cancer cells are sensitive to Etomoxir, Perhexiline (PMS) and Ranolazine (Rano).

FIG. 13 is an image and associated graph. Plated >10K cells/well (excess). MDV3100=enzalutamide=xtandi; Rano=Ranolazine. Excess cells were plated to ensure the effects observed were due to the treatment and not a very low number of plated cells, which tend to be very sensitive to drugs.

FIG. 14 is a pair of graphs showing that enzalutamide-resistant PCa cells grow better in the presence of enzalutamide compared to their parental controls. The parental controls are the left bar and the enzalutamide-resistant PCa cells are the right bar in each replicate of both graphs.

FIG. 15 is a pair of graphs showing the sensitivity of enzalutamide-resistant VCaP cells to perhexiline. Enzalutamide=MDV3100=xtandi.

FIG. 16 is a set of four graphs split over two pages. (A) Upper graph on first page of FIG. 16 graphs: This shows the effect of perhexiline (PMS, first bar within each set/replicate of bars), enzalutamide (MDV3100, second bar within each set of bars) and their combination (PMS/MDV, third bar within each set of bars) on the growth of LNCaP cells over 72 hours. The X-axis shows the concentrations (microMolar—μM) of PMS (first number in each set of three bars) and MDV (second number). Data is normalized to the vehicle treatment (no drugs). The Y-axis shows the normalized growth of cells using a colorimetric assay (MTS). (B) Lower graph on first page of FIG. 16 graphs: This shows the effect of Ranolazine (Rano, first bar), enzalutamide (MDV3100, second bar) and their combination (RANO/MDV, third bar) on the growth of LNCaP cells over 72 hours. The X-axis shows the concentrations (microMolar—μM) of RANO (first number) and MDV (second number) (e.g. “150-10”). Data is normalized to the vehicle treatment (no drugs). The Y-axis shows the normalized growth of cells using a colorimetric assay (MTS). (C) Upper graph on second page of FIG. 16 graphs: This shows the effect of perhexiline (PMS, first bar), enzalutamide (MDV3100, second bar) and their combination (PMS/MDV, third bar) on the growth of MDV-resistant-LNCaP (LNCaPMDVres) cells over 72 hours. The X-axis shows the concentrations (microMolar—μM) of PMS (first number) and MDV (second number). Data is normalized to the vehicle treatment (no drugs). The Y-axis shows the normalized growth of cells using a colorimetric assay (MTS). * p<0.05 combination (5 uM PMS+10 uM MDV) compared to individual treatments. (D) Lower graph on second page of FIG. 16 graphs: This shows the effect of Ranolazine (Rano, first bar), enzalutamide (MDV3100, second bar) and their combination (RANO/MDV, third bar) on the growth of MDV-resistant-LNCaP cells (LNCaPMDVres) cells over 72 hours. The X-axis shows the concentrations (microMolar—μM) of RANO (first number) and MDV (second number). Data is normalized to the vehicle treatment (no drugs). The Y-axis shows the normalized growth of cells using a colorimetric assay (MTS). *p<0.05 combination (150 uM Rano+10 uM MDV) compared to individual treatments.

FIG. 17 is a pair of graphs showing the effects of treatment on the indicated cells. (A) Upper Graph: This shows the effect of perhexiline (PMS, first bar within each set/replicate of bars), enzalutamide (MDV3100, second bar within each set of bars) and their combination (PMS/MDV, third bar within each set of bars) on the growth of LNCaPMDVres PMS Enza cells over 72 hours. The X-axis shows the concentrations (microMolar—μM) of PMS (first number) and MDV (second number). Data is normalized to the vehicle treatment (no drugs). The Y-axis shows the normalized growth of cells using a colorimetric assay (MTS). (B) Lower Graph: This shows the increased sensitivity of the MDV-resistant-LNCaP (LNCaP-Enza-Res) cells to the Ranolazine+Enza combination compared to the parental LNCaP cells. The Y-axis shows the relative growth over 72 hours. The X-axis shows the concentration of combination treatments for the Rano (first number) and enzalutamide (second number). Both concentrations are in microMolar (μM). The arrow shows how the 150 uM Rano+10 uM Enza combination results in more than 60% decrease in the LNCaP-Enza-Res (second, dark grey bar) compared to the control LNCaP parental cells (first, light grey bar).

FIG. 18 is a graph showing the sensitivity of the MDV-resistant-LNCaP (LNCaP-Enza-Res) cells to the Perhexiline+Enza combination. The Y-axis shows the relative growth over 72 hours. The X-axis shows the combination treatments for the Perhexiline (first number) and enzalutamide (second number). Both concentrations are in microMolar (μM).

FIG. 19 is a graph showing that LNCaP cells deficient in CPT1A expression (clone 79, second bar) are more sensitive to enzalutamide treatment compared to the control clone (clone 3, first bar). The x-axis shows the concentrations (microMolar—μM) for enzalutamide. The y-axis shows the normalized growth of cells over 72 hours. Data is normalized to the vehicle (0 μM, no drug) treatment. Data represents mean±SD.

FIG. 20 is a graph showing that nude mice (in vivo) bearing tumors of human cells that are resistant to enzalutamide (MDV) show signs of sensitivity to ranolazine. Injections of ranolazine started on day 5.

FIG. 21 is a pair of images (A and B) and three graphs (C, D and E) showing that CPT1A expression is increased in advanced prostate cancer using data gathered from human biopsies. FIGS. 21A and B show representative images of serial sections of benign and cancer tissue (arrows) from the same RRP specimen stained with H&E (A) or CPT1A (B) specific stain. (C) Quantification of CPT1A stain in 50 RRP specimens (11 benign and 39 cancer). Scoring was done by two persons. *p<0.05, AU=arbitrary units. (D) Graphical representation of Oncomine data (Setlur dataset) showing increased expression of CPT1A with advanced Gleason score. (E) Graph from cBioPortal showing CPT1A gene amplification in neuroendocrine (NEPC) and adenocarcinoma (SUC2C) samples in 2 recent datasets (Trento/Cornell/Broad 2016 and stand-up-2-cancer/PCF projects, respectively). The upper-most portion of both bars represents amplification (i.e. from ˜1% to ˜23% in the NEPC bar and ˜3.5% to 10.5% in the SUC2C bar). The lower portion (i.e. 0 to ˜1%) of the NEPC and the middle portion (i.e. ˜0.5% to ˜3.5%) of the SUC2C bar represents mutation. The lowest portion (i.e. 0 to ˜0.5%) of the SUC2C bar represents multiple alterations.

FIG. 22 is a set of five graphs based on in vitro data showing that the combination of fat oxidation inhibitors and enzalutamide results in a synergistic decrease of PCa growth. (A) LNCaP-enzalutamide resistant cells can grow in the presence of enzalutamide (MDV), ANOVA for MDV-resistant cells p<0.001, Post hoc *p<0.001 compared to parental cell line for each drug dose. (B) Increased sensitivity to the combination of ranolazine (Rano), etomoxir (Etom) or perhexiline (PMS) with enzalutamide in LNCaP-enzalutamide resistant cells, post hoc tests * p<0.05 compared to vehicle. (C-D-E) Increased sensitivity to the combination of etomoxir (C), perhexiline (D), or ranolazine (E) with enzalutamide in 22Rv1 cells, post hoc tests *p<0.001 compared to all other treatments. MDV=Enzalutamide=Xtandi

FIG. 23 is a set of three graphs demonstrating that mouse TRAMPC1 cells show increased sensitivity to the combination of beta-oxidation inhibitors and enzalutamide. (A-C) Clonogenic assay showing the effects of the combination of ranolazine (A), etomoxir (B) or perhexiline (C) with enzalutamide (MDV) in mouse TRAMPC1 cells, post hoc tests * p≤0.03 compared to individual treatments. These graphs illustrate the effect of the combinations in TRAMPC1 cells, which are mouse PCa cells that mimic the advanced PCa seen in patients. These cells are important model because they can be placed in syngeneic C57B1 mice with a complete immune system.

FIG. 24 is a pair of graphs showing that systemic ranolazine and enzalutamide combination treatment results in decreased tumor growth in vivo over 21 days. (A) Tumor growth in mice treated with vehicle (0.5% methyl cellulose with 0.1% tween 80), enzalutamide (20 mg/kg), ranolazine (40 mg/kg) or a drug combination (enza+rano) via gavage over 21 days. Five mice with 2 tumors each were used for each treatment group. Tumors were made of 22Rv1 cells implanted in the flanks of male nude mice. Arrow indicates beginning of treatment. Repeated measures ANOVA showed significant changes due to the effect of treatment over time (p<0.001, F=2.5 df=24). * p<0.05 (paired t-test comparing control to combination treatment group). (B) Effect of treatments on mouse body weights. No significant changes were observed and no signs of toxicity were observed throughout the study. These studies used human 22RV1 cells, which grow in nude mice without T cells.

### DETAILED DESCRIPTION OF THE PREFERRED EMBODIMENT

Prostate cancer (PCa) is the most common malignancy among western men and the second leading-cause of cancer related deaths. For men who develop metastatic, castration-resistant PCa (mCRPC), survival is limited, making the identification of novel therapies for mCRPC critical. Deficient lipid oxidation via carnitine palmitoyltransferase (CPT1) results in decreased growth and invasion, underscoring the role of lipid catabolism to fuel PCa growth. In fact, the CPT1A isoform is abundant in PCa, especially in those with high-grade tumors. Lipid oxidation can be stimulated by androgens. The synergistic effects of combining CPT1A inhibition and anti-androgen therapy is evaluated herein.

To better understand the clinical implications, fat oxidation inhibitors (etomoxir, ranolazine and perhexiline) in combination with anti-androgen therapy, such as treatment with enzalutamide, are evaluated in PCa cell models herein. A robust inhibitory effect of the combination is observed, including in enzalutamide-resistant cells and mouse TRAMPC1 cells, a more neuroendocrine PCa model. Lastly, using a xenograft mouse model, decreased tumor growth with a systemic combination treatment of enzalutamide and ranolazine is shown herein. In conclusion, the results shown herein demonstrate that improved anti-cancer efficacy can be achieved by co-targeting the AR axis and fat oxidation via CPT1A, which has important clinical implications, especially in the mCRPC setting.

The present invention provides compositions and methods for treating prostate cancer, such as anti-androgen drug-resistant prostate cancer and castration-resistant prostate cancer. The compositions include pharmaceutical compositions that block or reduce lipid metabolism, such as ranolazine, perhexiline, etomoxir, in combination with compositions providing anti-androgen therapy. Similarly, the invention includes methods of administering effective amounts of a lipid metabolism inhibitor, such as ranolazine, perhexiline, etomoxir, in combination with an anti-androgen drug, such enzalutamide, bicalutamide, abiraterone, galeterone, or AR-509, to treat patients having prostate cancer. The present invention also provides methods of enhancing the efficacy of prostate cancer drugs, including anti-androgen prostate cancer drugs.

Although new anti-androgen drugs like enzalutamide and abiraterone represent breakthroughs in the treatment of metastatic PCa, approximately 20-30% of patients have no response to these drugs. Furthermore, virtually all patients who respond initially acquire secondary resistance (Antonarakis, E. S., et al., 2014. N. Engl. J. Med. 371:1028-1038), that may be mediated by AR variants that keep the androgen-regulated programs working (Dehm, S. M., and Tindall, D. J. 2005. Expert. Rev. Anticancer Ther. 5:63-74), including the lipid metabolic program. Thus, the present invention seeks to target novel aspects of lipid metabolism in PCa, including those that are not only focused on lipogenesis, employing metabolic and anti-androgen drugs that synergize to effectively thwart castration-resistant-PCa growth.

The mechanisms by which PCa cells use lipids to their benefit are poorly understood. De novo fatty acid synthesis can occur in cancer cells from glucose, in a pathway largely controlled by the enzyme fatty acid synthase (FASN), and is associated with cell growth, survival and drug resistance (Swinnen, J. V., et al., 2006. Curr. Opin. Clin. Nutr. Metab Care 9:358-365; Suburu, J., and Chen, Y. Q. 2012. Prostaglandins Other Lipid Mediat. 98:1-10). However, the biochemical mechanisms governing the relationships between lipid synthesis, lipid utilization, and cancer growth remain largely unknown.

Overexpression of key enzymes in lipid synthesis in prostate cancer is characteristic of both primary and advanced disease (Vavere, A. L., et al., 2008. J. Nucl. Med. 49:327-334). Targeting lipid metabolism enzymes in PCa offers new avenues for therapeutic approaches.

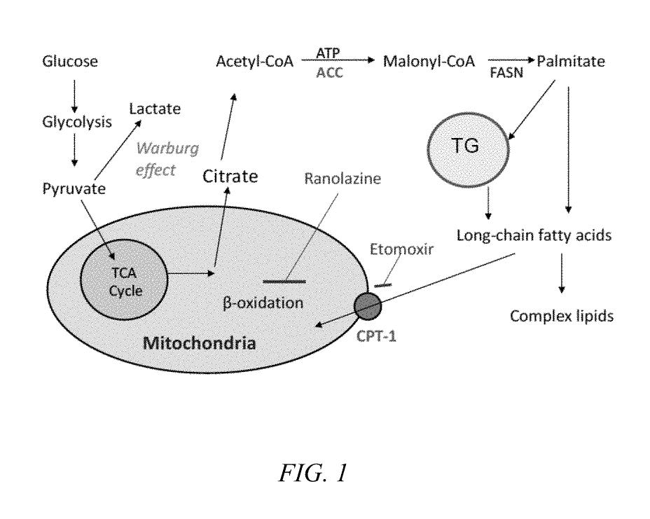

The role of lipid utilization (beta oxidation) can be demonstrated in a translational research manner using safe metabolic inhibitors that can be used in the lab and the clinic. Several lipid catabolism inhibitors, including etomoxir and ranolazine, are now available that show low toxicity and are implemented in the clinic swiftly (FIG. 1). Etomoxir is a safe irreversible inhibitor of the long chain fatty acid transporter and has been used in the treatment of heart failure (Abozguia, K., et al., 2006. Nat. Clin. Pract. Cardiovasc. Med 3:490-498). Etomoxir works by inhibiting carnitine-palmitoyl-transferase-1 (CPT-1) and blocking the entry of long chain fatty acids into the mitochondria for oxidation, forcing cells to use the oxidation of glucose for energy. Ranolazine is an FDA-approved drug known to reduce beta oxidation in the heart, but the mechanism of action is not as well defined as etomoxir (Lionetti, V., et al., 2011. Cardiovasc. Res. 90:202-209). Fatty acids (from diet or from lipid storage (TG)) can be used for fuel (via beta oxidation) and to promote growth, or used to generate lipid signaling molecules that shape the fate of the cell, like eicosanoids (Shappell, S. B., et al., 1999. Am. J. Pathol. 155:235-245; Tang, S. , et al., 2002. J. Biol. Chem. 277:16189-16201) and phospholipids (Sriburi, R., et al., 2004. J. Cell Biol. 167:35-41). Presently, there are no studies of these metabolic drug effects on prostate lipid metabolism and androgen action. However, as discussed and shown here, they are attractive tools for the mechanisms of lipid use in prostate cancer and in application for targeted therapies for PCa.

Blockade of the lipid oxidation via CPT1 leads to metabolic stress, due to a decrease ATP production (Zaugg, K., et al., 2011. Genes Dev. 25:1041-1051). This phenotype was associated with accumulation of lipid droplets. One of the consequences of toxic lipid accumulation is the development of endoplasmic reticulum (ER) stress, which is an alarm mechanism to try to restore the normal function of the ER; that is, the synthesis and processing of secretory and membrane proteins (Maly, D. J., and Papa, F. R. 2014. Nat. Chem. Biol. 10:892-901). Failure in the attempts to restore ER homeostasis usually result in cell death, including in cancer (Feldman, D. E., et al., 2005. Mol. Cancer Res. 3:597-60524-26; Holz, M. S., et al., 2013. Mol. Cancer Ther. 12:173-183). Studies from fatty-liver disease have shown that delivery of long chain saturated fatty acids to liver cells or increasing the amount of saturated fatty acids (like palmitate) within the liver provokes ER stress, apoptosis and liver injury (Wang, D., et al., 2006. Endocrinology 147:943-951-29). The present invention shows that the catabolism of fatty acids delivered to or stored within cells is an important determinant of ER homeostasis, apoptosis and disease progression.

As shown herein, the ability to burn lipid in the mitochondria is an important determinant of lipid and ER homeostasis and disease progression in prostate cancer. Therefore there is a need to elucidate how lipid burning capacity via CPT1A favors growth of PCa and to identify molecular mechanisms that link ER homeostasis, androgen action and apoptosis in prostate cancer models. The present invention addresses these important needs.

Although new anti-androgen drugs like enzalutamide and abiraterone represent breakthroughs in the treatment of castration-resistant prostate cancer, approximately 20-30% of patients have no response to these drugs. Furthermore, virtually all patients who respond initially acquire secondary resistance, likely mediated by mutated or modified androgen receptors that keep the androgen-regulated programs working, including the lipid metabolic program. The mechanisms by which prostate cancer cells use lipids to their benefit are poorly understood. Our studies, as shown herein, indicate that prostate cancer preferentially burn lipid in the mitochondria to obtain energy for growth. The entry of lipid into the mitochondria is mediated by the enzyme CPT1, which functions as a gatekeeper for the lipid. Accordingly, while not wishing to be bound by a theory, targeting the lipid burning capacity of prostate cancer, in combination with anti-androgen drugs, can result in castration-resistant-prostate cancer to be effectively stopped. Borrowing safe drugs from other fields, like obesity (etomoxir) and cardiovascular disease (ranolazine), provides effective combinatorial therapies that target two Achilles' heels of prostate cancer: androgen action and lipid burning-derived energy.

The present invention leverages a number of novel concepts, including the observation of lipid burning as a regulator of AR action and prostate cancer survival. At the intersection of lipid metabolism and androgen action, we have identified CPT1 as a metabolic enzyme needed to support prostate cancer growth and avoid lipotoxicity, ER stress and apoptotic death. This novel observation represents a molecular link between metabolism in the mitochondria and androgen action in the nucleus. The use of safe lipid inhibitors from other research fields (e.g. etomoxir, ranolazine) in combination with current anti-androgen drugs (e.g. enzalutamide) will block PCa growth and elucidates the underlying mechanisms that connect CPT1A to prostate cancer growth and androgen action.

Overview of the examples that follow: 1—Fat oxidation inhibitors decrease the viability of PCa cells in vivo. 2—CPT1A is abundant in high grade human PCa. 3—Etomoxir treatment increases ER stress markers in LNCaP cells. 4—Etomoxir and enzalutamide synergize to decrease PCa AR content and growth. 5—CPT1A-deficient cells (CPT1AKD) have increased sensitivity to enzalutamide. 6—The partial beta-oxidation inhibitor ranolazine decreases growth of enzalutamide-resistant cells and modifies AR content and action. 7-CPT1A genomic editing with CRISPR technology decreases PCa growth and ability to form colonies. 8—Mouse TRAMPC1 prostate cancer cells are sensitive to ranolazine and enzalutamide combination treatments. 9—Table listing of the cell lines used in the studies. 10—Investigation of CPT1A and enzalutamide interaction in viveo using mouse xenografts. 11—Investigation of fat oxidation inhibitors in enzalutamide-resistant cells in vivo. 12-Investigation of the mechanisms behind the intersection of CPT1A and androgen action in prostate cancer cell models.

### Example 1—Fat Oxidation Inhibitors Decrease the Viability of PCa Cells In Vivo

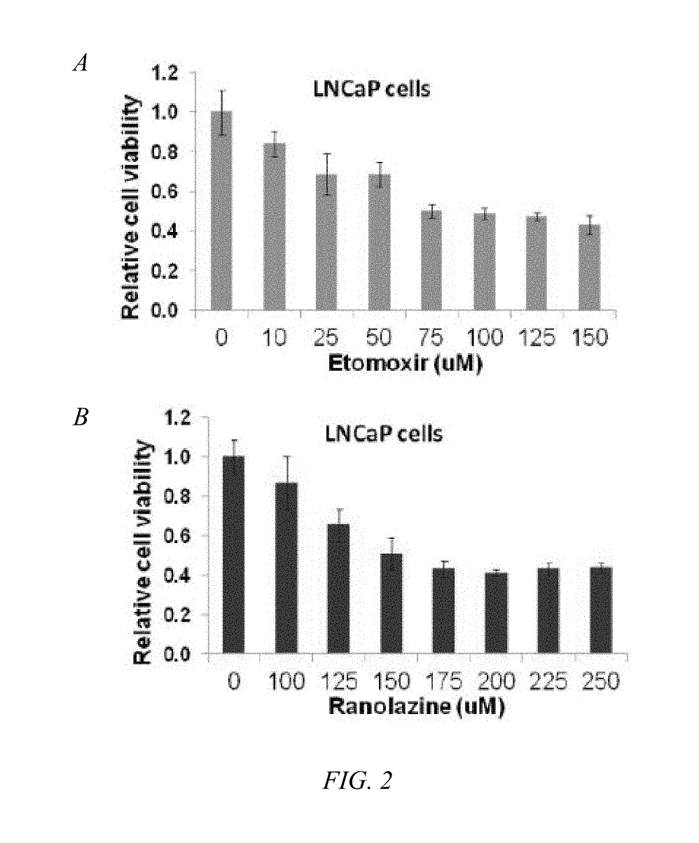

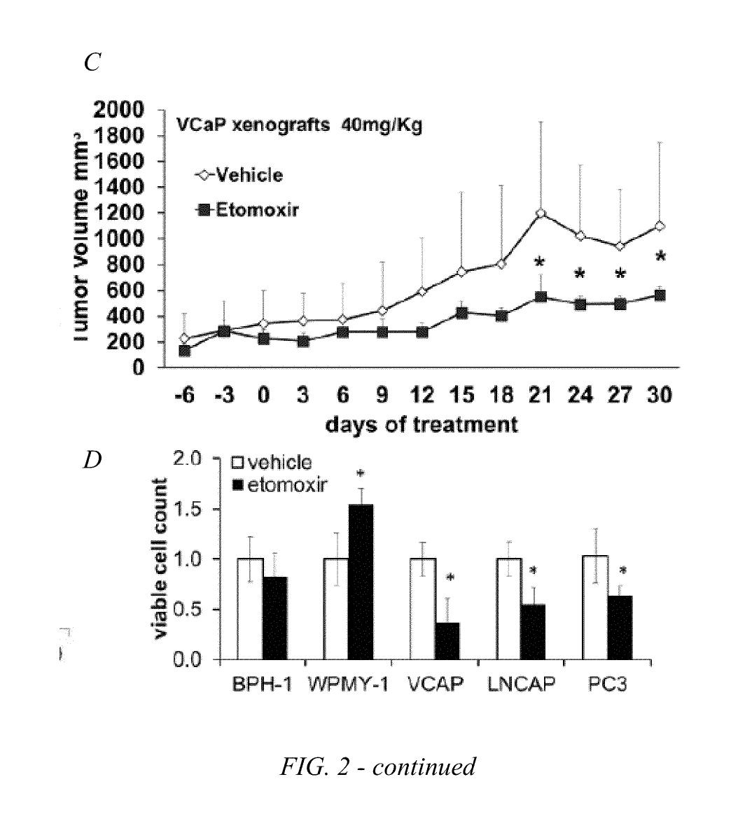

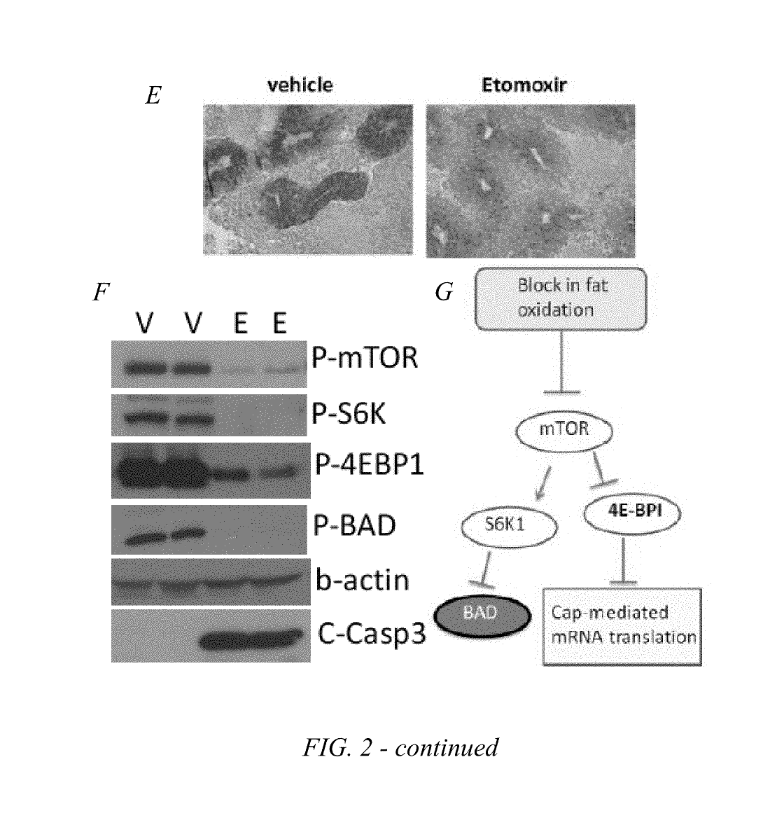

The clinically safe inhibitor etomoxir is able to block 14C-lipid oxidation in PCa cells and decrease their growth, an effect that was not seen in the benign BPH-1 and WPMY-1 cells. This growth effect was also observed in viva) using xenografts and treating mice with etomoxir (40 mg/kg/day) for 21 days (FIG. 2C). These effects were associated with increased palmitate-containing ceramides (not shown) and decreased mTOR and its downstream effectors S6K and 4EBP-1, leading to BAD de-phosphorylation (at S112) and apoptosi s.

### Example 2—CPT1A is Abundant in High Grade Human PCa

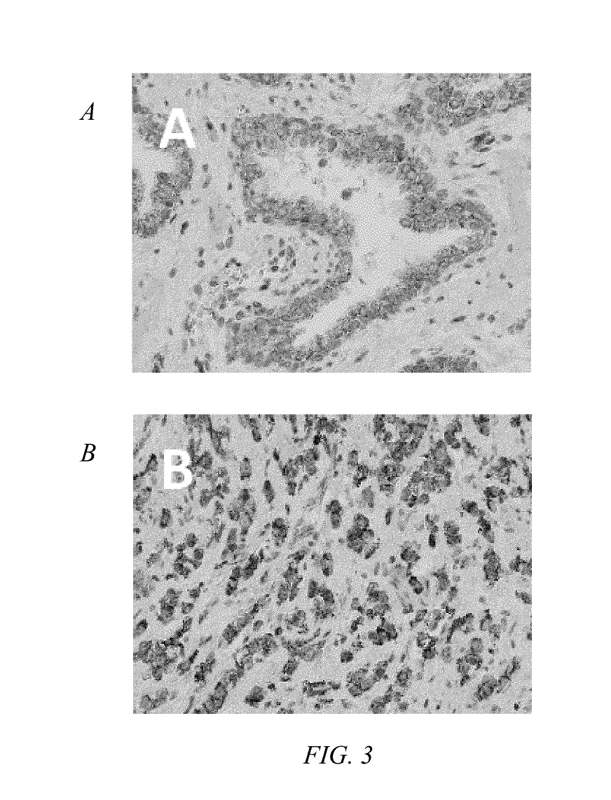

The effects of etomoxir in primary PCa cells derived from prostatectomies carried out at University of Colorado hospital were examined. It was observed that the cancer cells responded to the etomoxir treatment with a momentary increase in glucose uptake, but a concomitant decrease in viability was observed compared to their matched non-cancerous cells. These results indicated that our cell lines and mouse models can be translated to human PCa (data not shown). Additionally, human prostatectomy samples have been stained for CPT1A (CU Pathology Core) and it was observed to be more abundantly expressed in prostate cancer compared to normal, FIG. 3.

### Example 3—Etomoxir Treatment Increases ER Stress Markers in LNCaP Cells

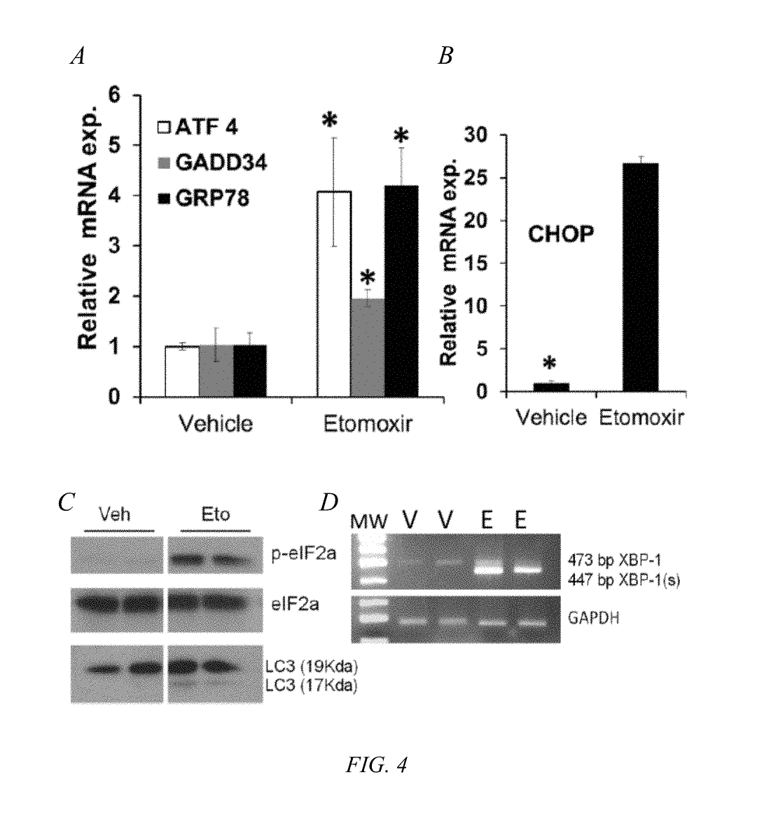

Changes in markers of ER stress were examined to elucidate the molecular mechanisms behind the decreased viability with etomoxir. Manipulation of lipid metabolism in PCa can result in ER stress. This is important since key nodal components of the ER stress pathway are druggable targets. Interestingly, ER stress negatively regulates mTOR and strong expression of transcription factor CHOP may be driven by palmitate accumulation. FIG. 4 shows the increase in markers of ER stress in etomoxir-treated LNCaP cells. The additional increase in autophagy markers (LC3) is likely a reflection of decreased mTOR action (Su, B., and Jacinto, E. 2011. Crit Rev. Biochem. Mol. Biol. 46:527-547).

### Example 4—Etomoxir and Enzalutamide Synergize to Decrease PCa AR Content and Growth

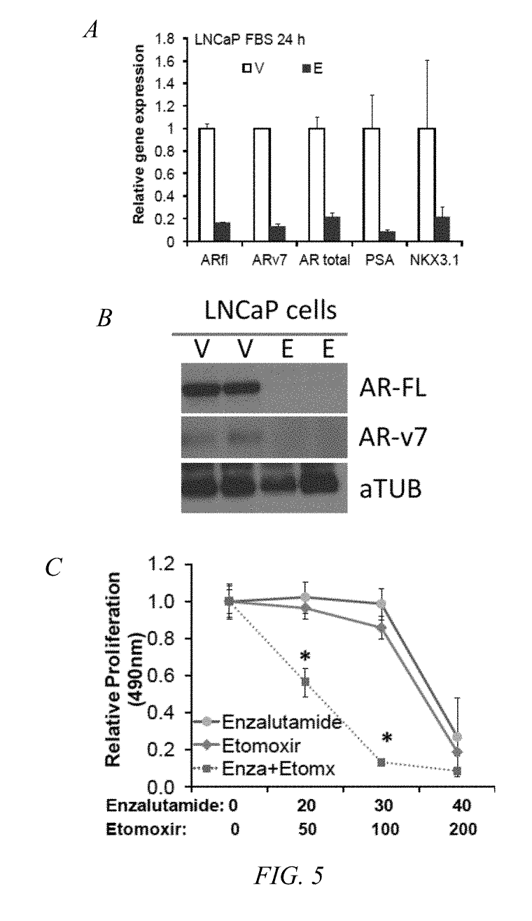

Since LNCaP and VCaP cells died with etomoxir, the expression of AR and its downstream targets (PSA and NKX3.1) was also examined. A significant decrease of AR and PSA mRNA has been observed (Schlaepfer, I. R., et al., 2014. Mol. Cancer Ther. 13:2361-2371). Studies were conducted to investigate if etomoxir and the anti-androgen enzalutamide could synergize in killing PCa cells. FIG. 5 shows that when the expression of androgen receptor (AR) and its variant forms associated with castration-resistance PCa was examined, a significant decrease after etomoxir treatment was observed, FIGS. 5A and 5B, indicating a strong connection between lipid oxidation and AR content and action. The combination of enzalutamide and etomoxir produced a significant synergistic effect on cell proliferation (FIG. 5C), that is probably driven by the strong decrease in androgen receptor and androgen action.

### Example 5—CPT1A-Deficient Cells (CPT1AKD) have Increased Sensitivity to Enzalutamide

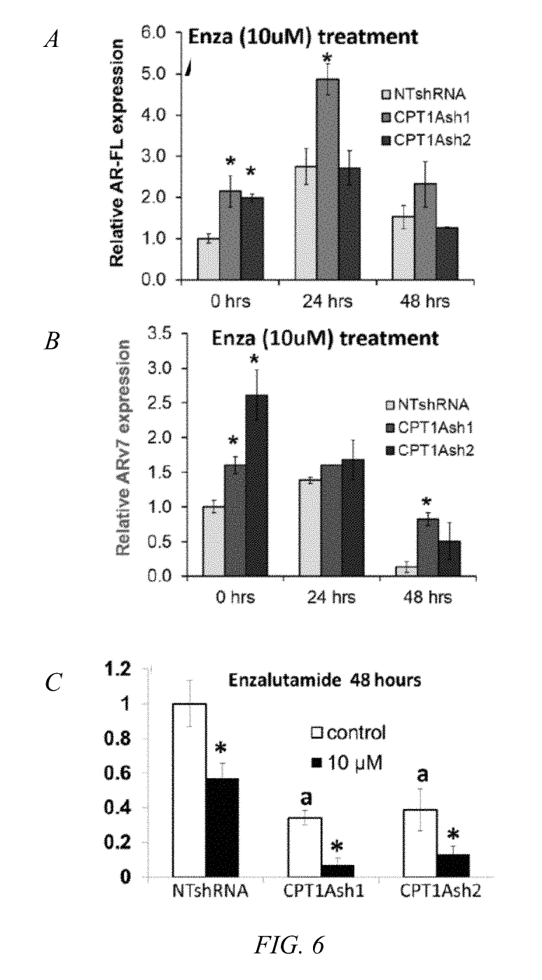

Etomoxir targets CPT1. CPT1A was stably knocked down in LNCaP cells to examine its effects on fat oxidation, cell growth and synergism with enzalutamide. Interestingly, making these clones was challenging, since growth with decreased CPT1A expression was compromised. These CPT1AKD clones, like the treatment with etomoxir, also showed a synergistic effect on cell growth when treated with enzalutamide (MDV3100) (FIG. 6). However, an increase in AR full length and ARv7 variant was observed with enzalutamide treatment (FIGS. 6A & 6B). This AR upregulation in response to CPT1A knockdown increases the sensitivity of the clones to enzalutamide. It is possible that the decrease in AR observed with etomoxir is due to off-targets effects of the drug (besides CPT1), and/or the cells may need to be studied at earlier time points when apoptotic and ER stress mechanisms have not been set in motion.

### Example 6—the Partial Beta-Oxidation Inhibitor Ranolazine Decreases Growth of Enzalutamide-Resistant Cells and Modifies AR Content

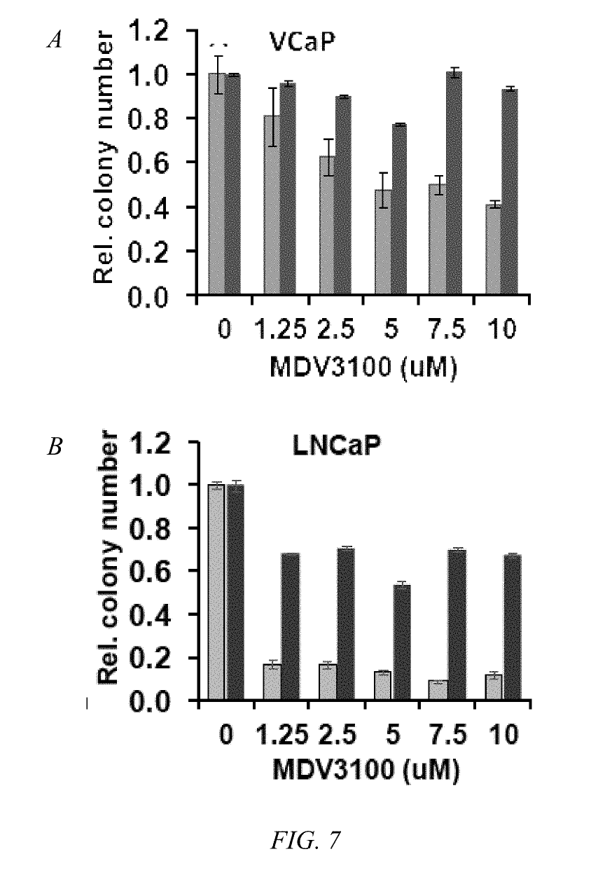

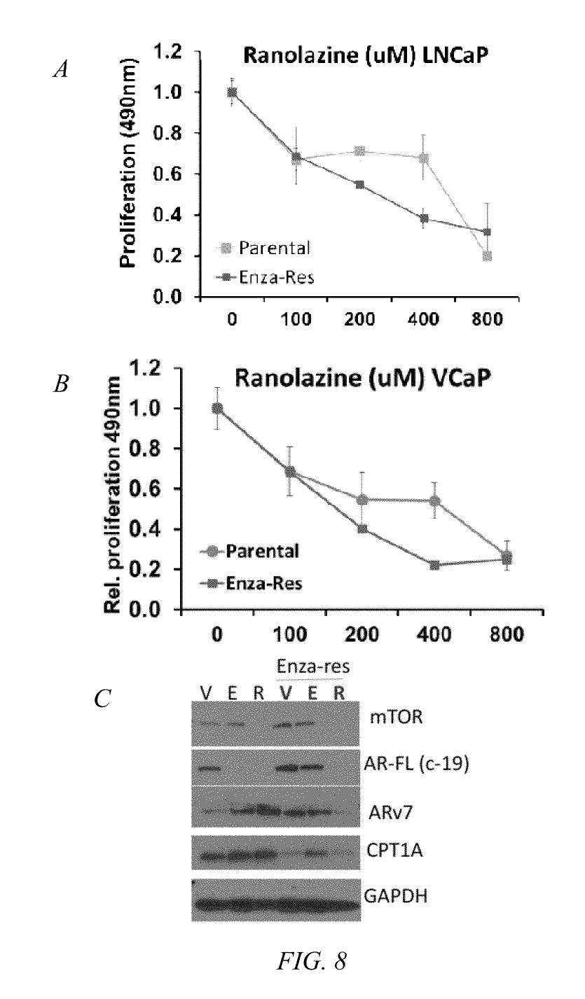

Despite advances in anti-androgen therapy, resistance to enzalutamide is frequent and responsible for treatment failure. Thus, CPT1A content and AR expression in enzalutamide-resistant PCa cells was examined, particularly, LNCaP-Enza-Res and VCaP-Enza-Res lines (FIG. 7) which were generated by growing them in media with enzalutamide for 6 months. FIG. 8 shows that enzalutamide-resistant LNCaP and VCaP cells are more sensitive to ranolazine than their corresponding parental controls. Parental LNCaP and VCaP cells treated with vehicle were used as their corresponding controls.

Etomoxir treatment produced similar results. Protein analysis of VCaP cells showed that ranolazine (R) effectively decreases AR full length (AR-FL) and ARv7 in VCaP-Enza-Res cells. CPT1A expression appears negatively correlated with AR-FL but positively correlated with ARv7 (FIG. 8C).

### Example 7—CPT1A Genomic Editing with CRISPR Technology Decreases PCa Growth and Ability to Form Colonies, that is, CPT1A is Needed to Maintain the Clonogenicity of the Prostate Cancer Cells

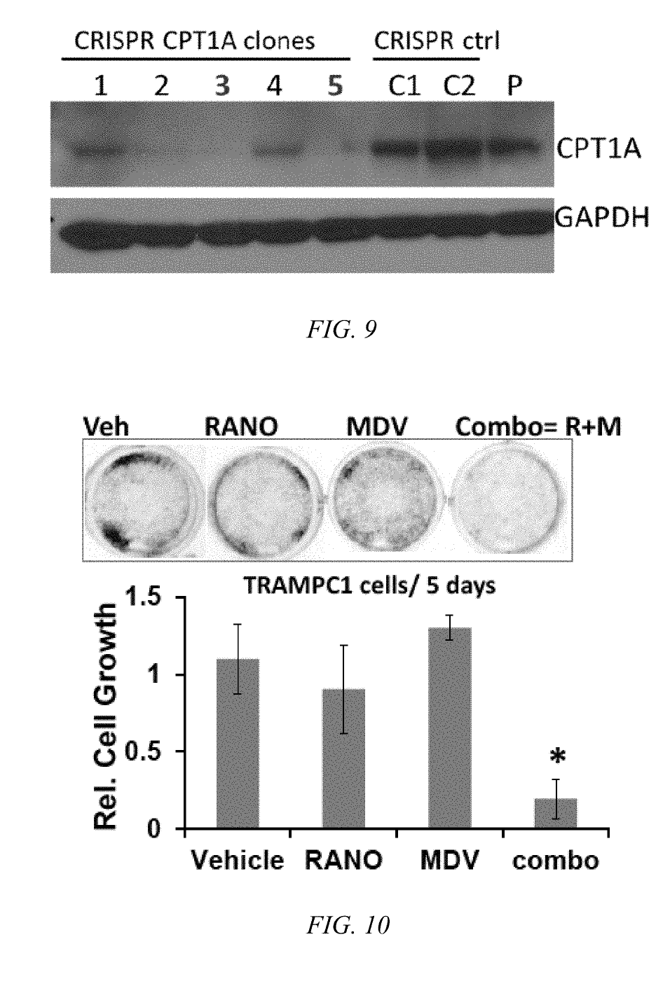

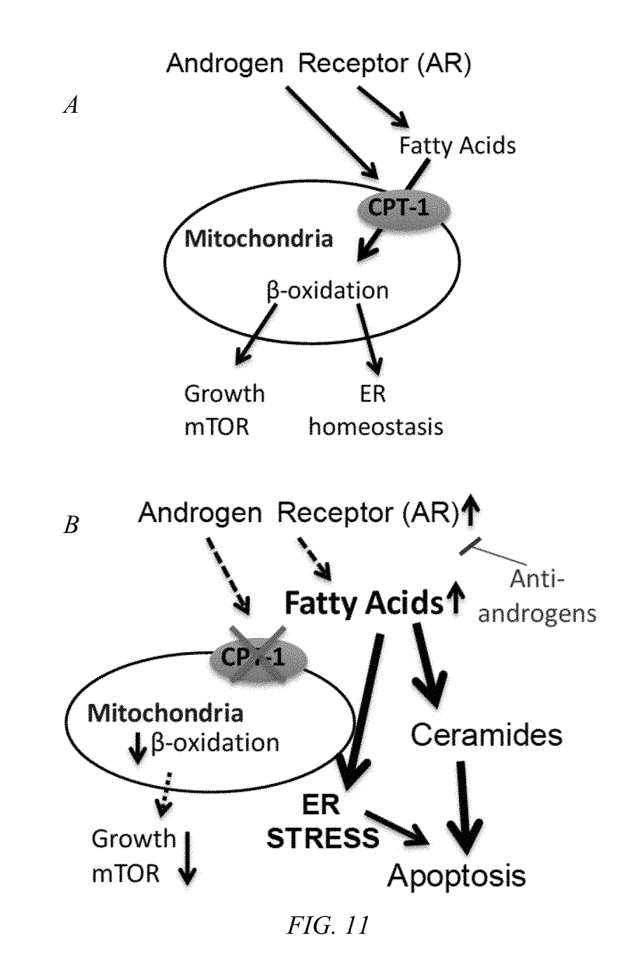

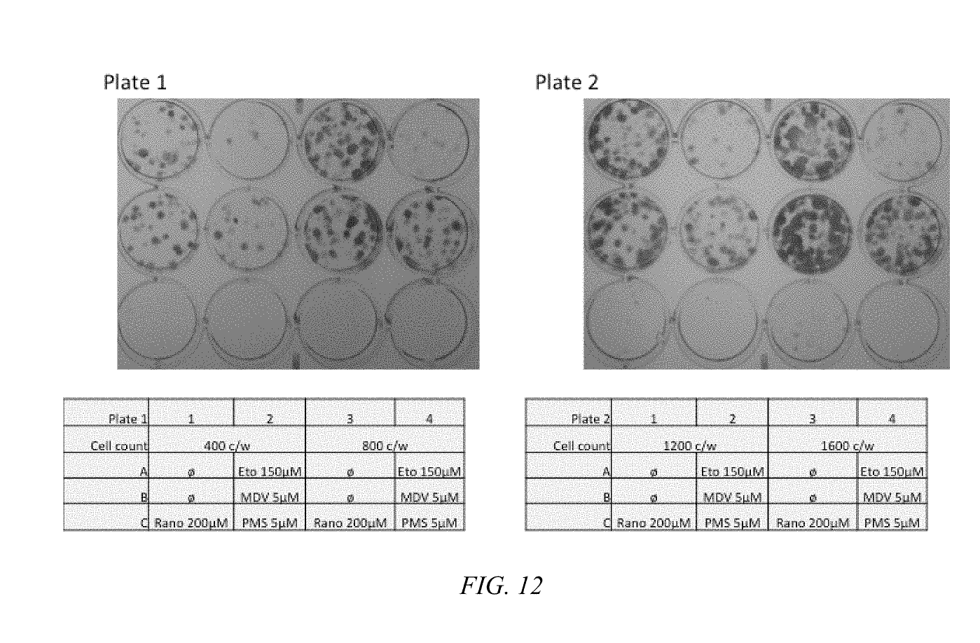

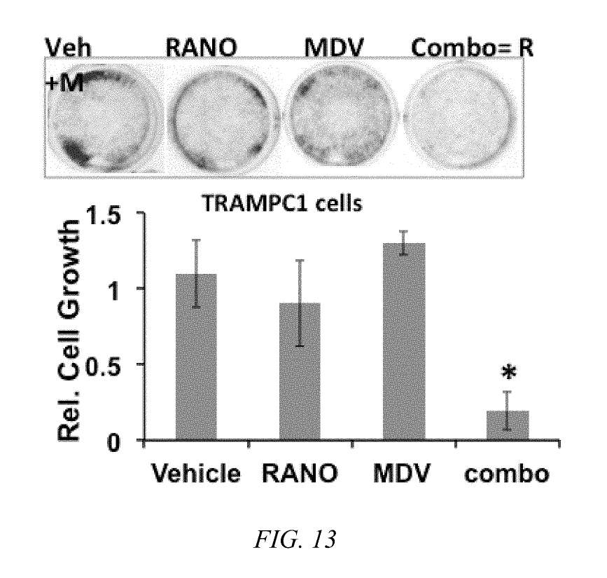

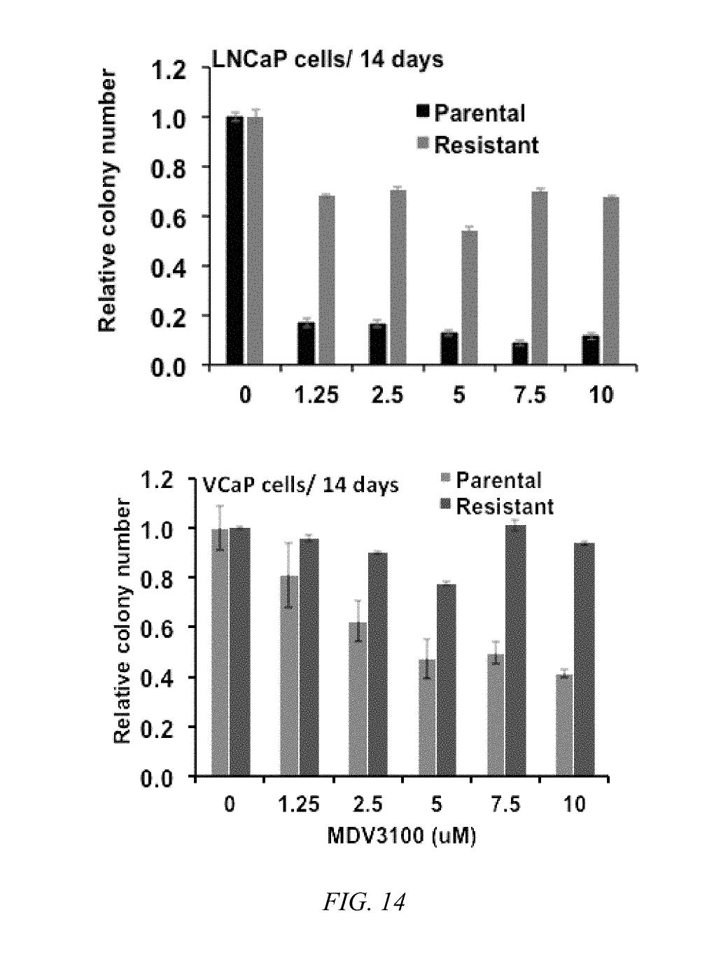

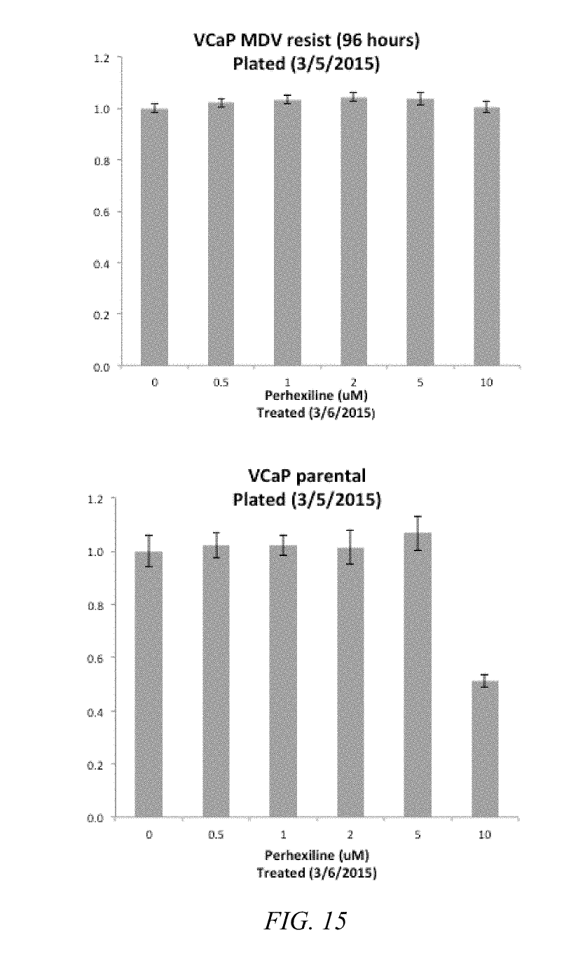

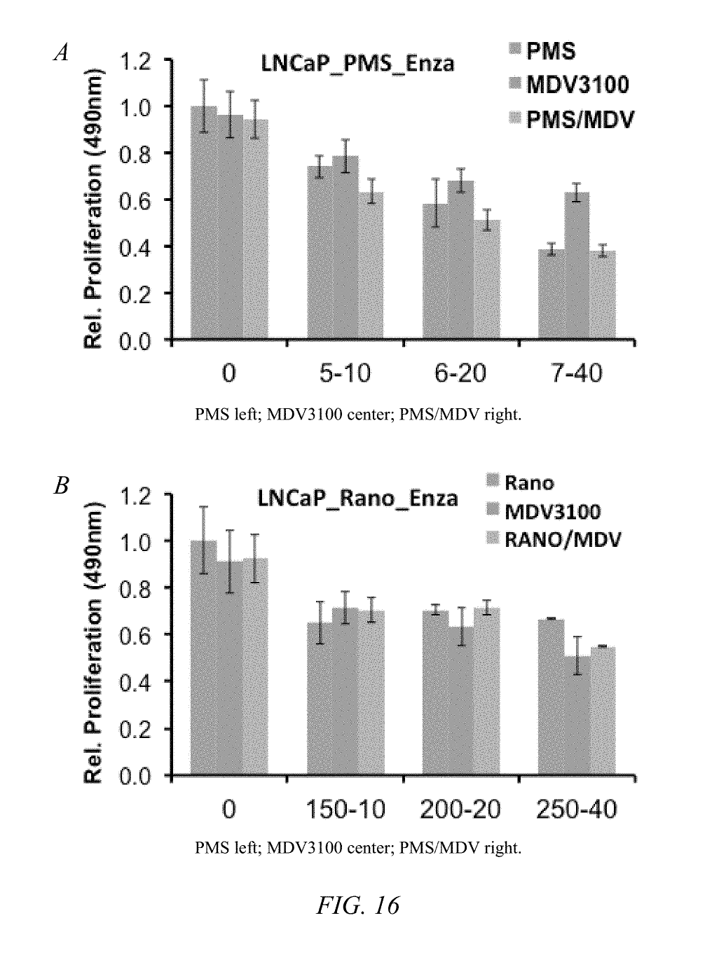

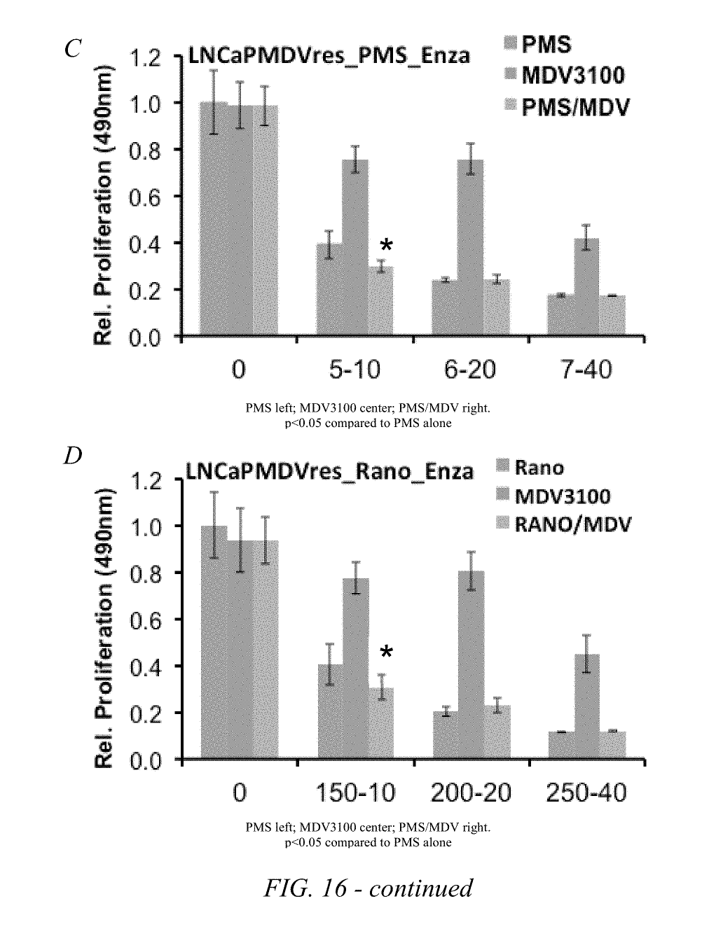

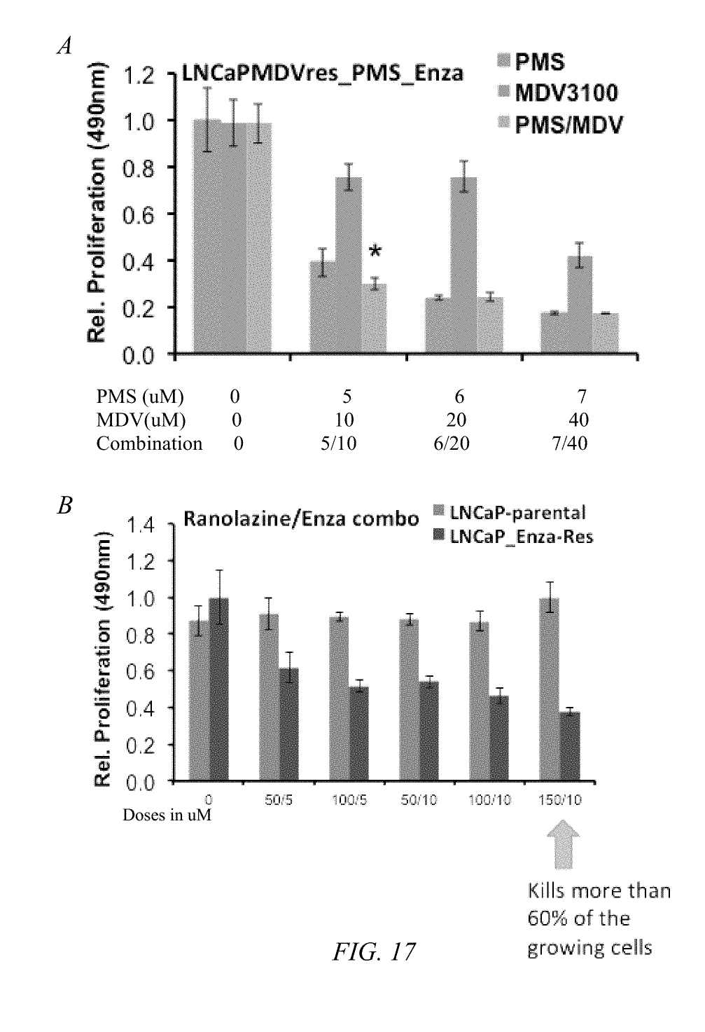

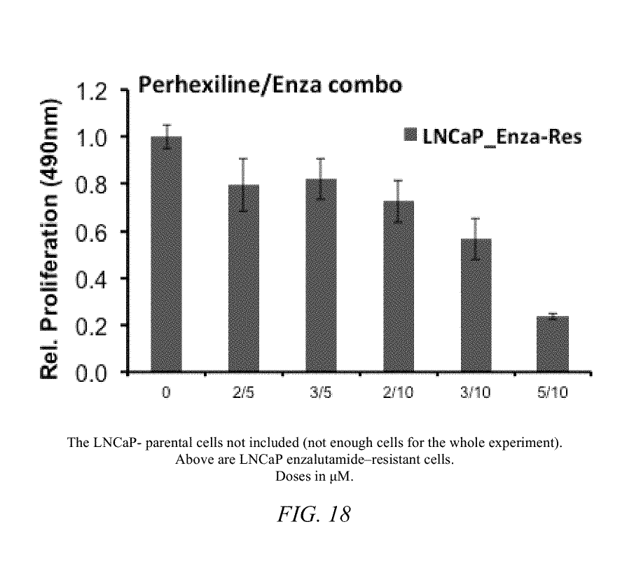

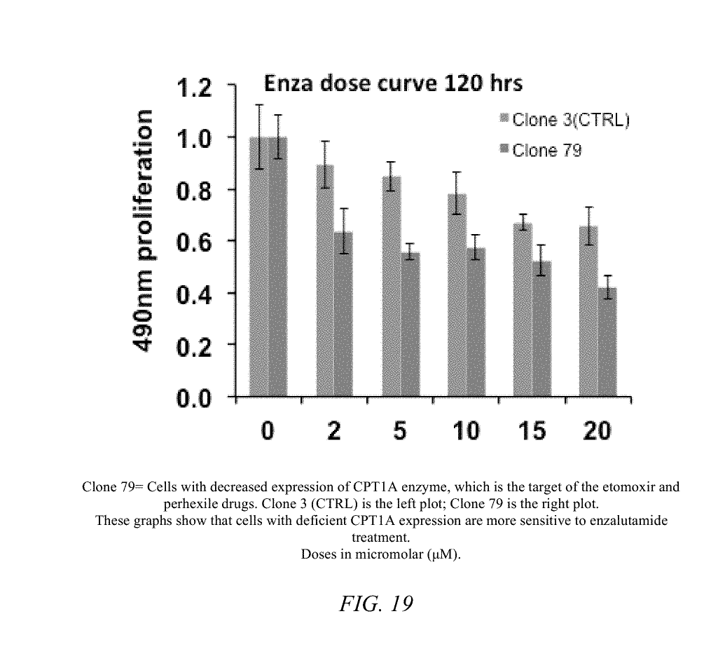

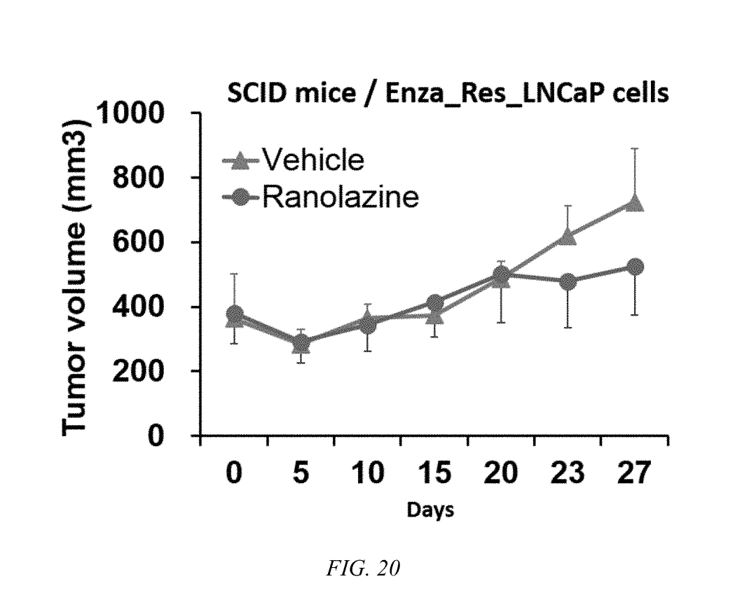

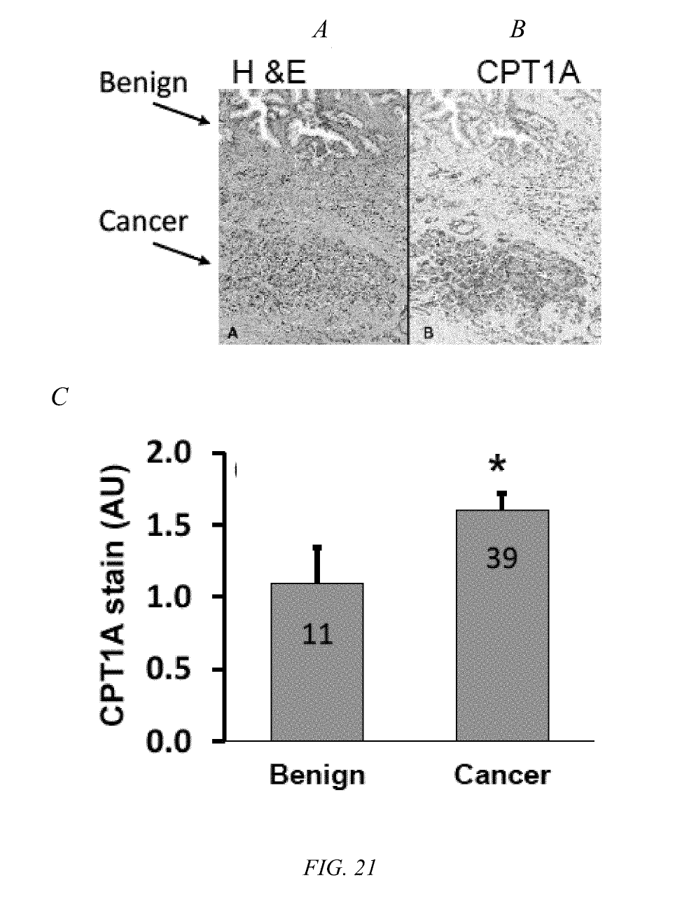

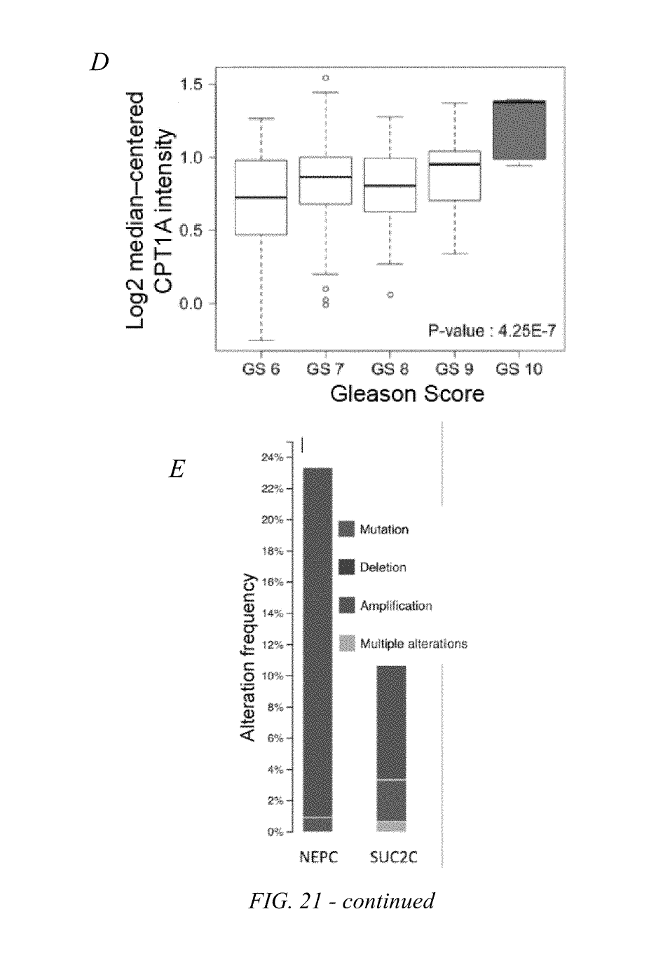

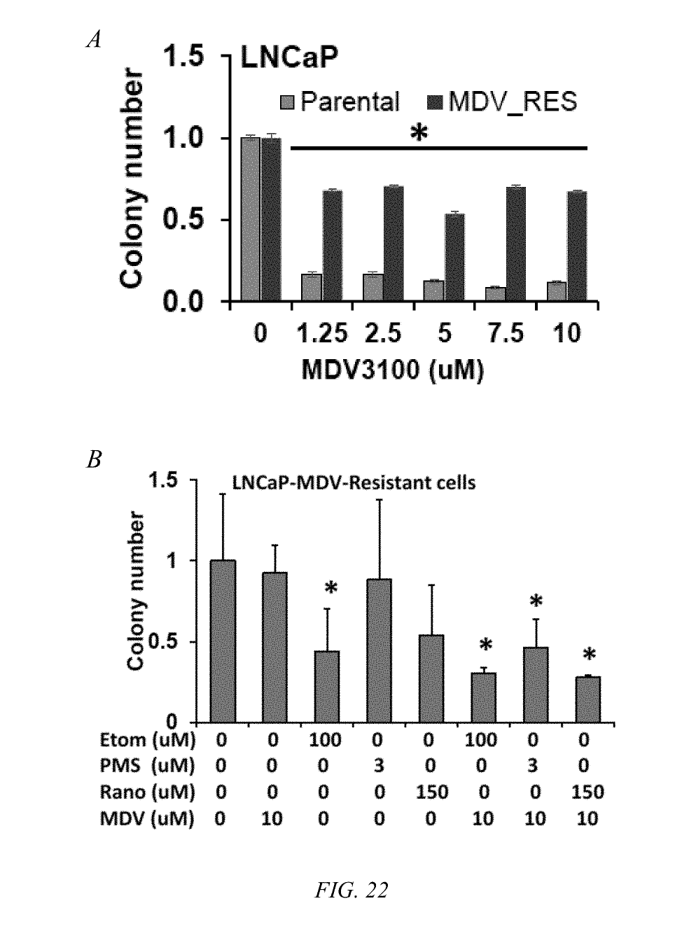

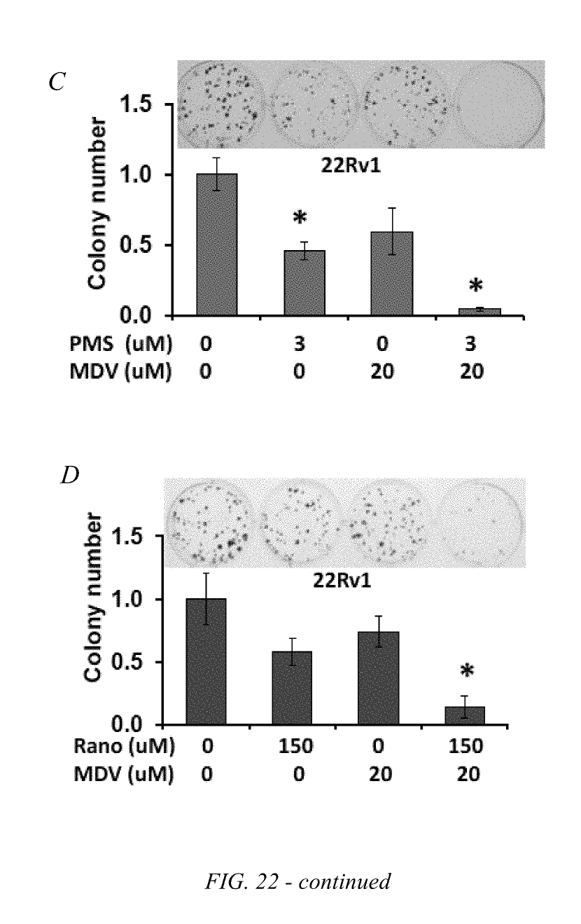

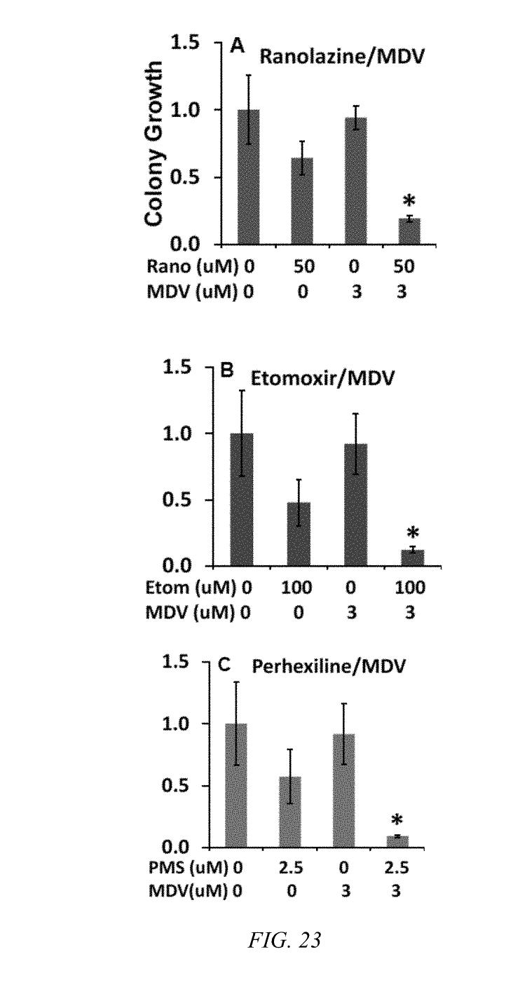

Since CPT1A KD clones are still expressing some level of enzyme, the new genomic editing technique called Clustered Regularly Interspaced Short Palindromic Repeats (CRISPR) has been employed inactivate the CPT1A gene in LNCaP cells. This methodology can be used to investigate the response to anti-androgens (FIG. 9). FIG. 9 shows that the CRISPR technology specifically targeting CPT1A gene, is able to reduce the expression of CPT1A in LNCaP cells. The C1 and C2 clones were also subjected to CRISPR technology protocol, but without specific targeting to any human gene (scramble) so they do not show any decrease in CPT1A expression. Thus, this photo shows the specificity of the CPT1A-CRISPR approach used in these studies.

### Example 8—Mouse TRAMPC1 Prostate Cancer Cells are Sensitive to Ranolazine and Enzalutamide Combination Treatments

Additionally, the mouse cell line (TRAMPC1) has been tested for its sensitivity to ranolazine, enzalutamide (MDV3100) and their combination. This cell line from mouse PCa allows the evaluation of treatments in vivo, using immune-competent C75BL/6 mice, which are syngenic with the TRAMPC1 cells. (FIG. 10) FIG. 10 shows that TRAMPC1 cells are sensitive to the combination of Ranolazine (RANO) and enzalutamide (MDV). They are not very sensitive to each drug treatment alone but the combination produces a significant decrease in growth (more than 50%) compared to vehicle treatment (no drugs) or each individual treatment. This is important because the doses of drugs used here are very low and non-toxic, but the combination of these low doses is very powerful inhibiting cell growth.

### Example 9—Table Listing of the Cell Lines Used in the Studies

TABLE 1
Cell Model

| | Description |
|---|---|
| CPT1A knock-down | LNCaP cells with shRNAs specific to CPT1A and non- |
| cells | targeting shRNA for control, (Schlaepfer, I. R., et al., |
| | 2015. Mol. Imaging Biol. 17: 529-538; Schlaepfer, I. R., et |
| | al., 2014. Mol. Cancer Ther. 13: 2361-2371.). These cells |
| | can be used to study AR content, action and lipid |
| | metabolism pathways. |
| CPT1A knock-out | LNCaP cells with genetic deletion of CPT1A (mono- and |
| cells | bi-allelic deletion) using CRISPR technology. These |
| | genetically-edited cells lack feedback responses in |
| | CPT1A expression and provide a clean background to |
| | study AR and the effects of CPT1A reconstitution |
| | (wildtype and mutated cDNA) in the PCa cells. |
| Enzalutamicle (Enza)- | Parental and Enza-resistant LNCaP and VCaP cell |
| resistant cells | models. These clones are labeled with luciferase reporter |
| | for in vivo tracking with bioluminescence. Since they |
| | have decreased levels of CPT1A expression compared to |
| | their naïve counterparts, they facilitate the study ligand- |
| | independent AR. Reconstitution of CPT1A expression |
| | can be used to evaluate re-sensitization to anti-androgens. |
| PC3-Luciferase (PC3- | PC3-LUC cells labeled with luciferase. They serve as a |
| LUC) | model of AR negative prostate cancer, (Schlaepfer, I. R., |
| | et al., 2015. Mol. Imaging Biol. 17: 529-538). These cells |
| | do not express AR or PSA but express low levels of |
| | CPT1A so they can be used as control cells for the |
| | mechanistic studies. |
| TRAMP-C1 cells | Mouse prostate cancer cell model that is syngenic with |
| (mouse) | C57BL/6 mice so immune-competent studies in vivo can |
| (parental and cpt1aKD) | be done (Foster, B. A., et al., Cancer Res. 57: 3325-3330). |
| | This model has been used for enzalutamide studies |
| | (Lin, T. H., et al., 2013. Cell Death. Dis. 4: e764). The |
| | TRAMP-C1 cpt1aKD cells are currently under |
| | puromycin selection. |

### Example 10—Investigation of CPT1A and Enzalutamide Interaction In Vivo Using Mouse Xenografts

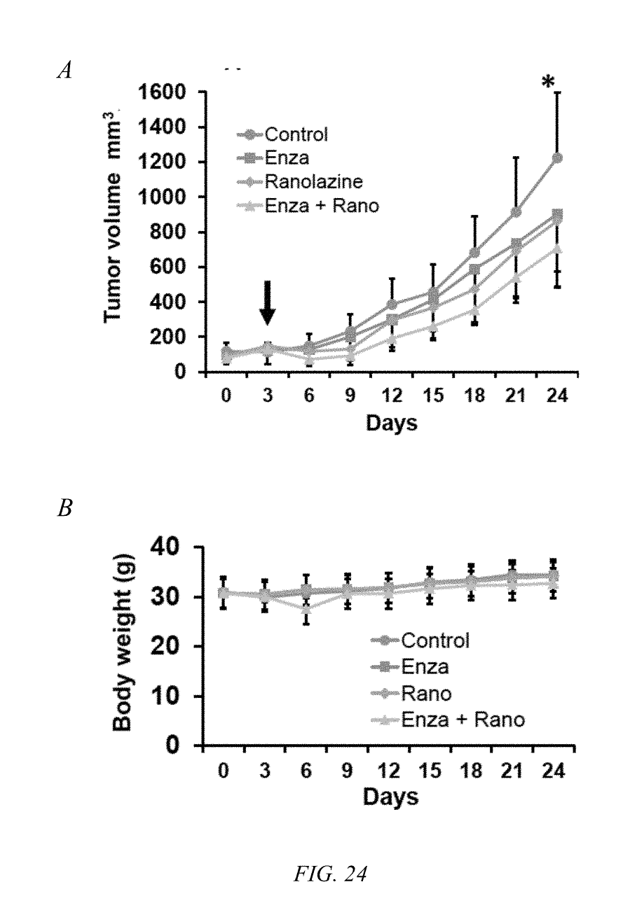

CPT1A and enzalutamide interaction has been investigated in vivo using mouse xenografts (See e.g. FIG. 24). Lipid oxidation via CPT1 supports the proliferation of PCa cells and disruption of this lipid oxidation leads to death of PCa cells. An increase in AR full-length content associated with decreased growth was observed in LNCaP-CPT1A-KD cells, making the cells more sensitive to enzalutamide. A combination of decreased beta-oxidation via shCPT1A with systemic enzalutamide will result in a synergistic decrease in tumor growth, likely driven by lipotoxicity leading to apoptosis. SCID (for the human cells) and immune-competent mice (for the TRAMPC1 cells) can be used to demonstrate the effect to human PCa. Furthermore, CPT1A is important in the maintenance of tumor tolerance in T-cells (Patsoukis, N., et al., 2015. Nat. Commun. 6:6692).

Male athymic SCID mice can be injected subcutaneously with 2 million LNCaP-Control or LNCaP-CPT1A-KD cells suspended in Matrigel (BD Biosciences). After palpable tumors are observed, intraperitoneal drug treatment (vehicle or enzalutamide: 10 mg/Kg/day) begins and lasts for 4 weeks. These doses of drugs have been safely used in mice with positive outcomes (Toren, P., et al., 2014. Eur. Urol.). Likewise, TRAMPC1-control and TRAMPC1-cpt1aKD cells can be used for the generation of xenografts in the C57BL/J6 mice and subsequent treatments.

Each mouse receives 2 grafts. Eight mice (16 grafts) can be used per treatment group plus 4 mice for unexpected deaths, totaling 18 mice per cell line. Thus, 36 (18×2 treatments) nude mice can be used (see statistical section below). In parallel, 36 C57BL/6 mice will be used for the TRAMPC1 grafts.

Tumor growth and specimen collection: Tumor volumes can be measured daily with calipers as described (Schlaepfer, I. R., et al 2014. Mol. Cancer Ther. 13:2361-2371). Serum PSA can be measured once a week by enzymatic immunoassay (Abbott IMX). During the time of growth of the xenografts, urine samples of the mice can be collected to assess the eicosanoid species produced, since they have been shown to increase with prostate gland disease (Nithipatikom, K., et al., 2006. Cancer Let. 233:219-225).

Effectiveness of CPT1A knockdown in vivo: Expression of CPT1 isoforms can be determined by western blot of tumor lysates (Schlaepfer, I. R., et al 2014. Mol. Cancer Ther. 13:2361-2371). qRTPCR can be used to measure CPT1A expression.

Androgen receptor action: qRTPCR can be used to analyze PSA, NKX3.1, AR-full length and the common variants ARv7 and ARv567 as described (Schlaepfer, I. R., et al., 2014. Mol. Cancer Ther. 13:2361-2371). Serum PSA can be measured in serum at the end of study by enzymatic immunoassay (Abbott IMX).

Effectiveness of therapy and assessment of resistance: The problem in metastatic PCa is resistance to therapy. Because of the observed increase in AR variants with decreased CPT1A expression (the ones likely responsible for the resistance to therapy), it is possible that the enzalutamide therapy works at the beginning (because full-length AR is also increased), but resistance arises in the CPT1AKD tumors. Additionally, hypoxia status of tumors is strongly correlated with aggressiveness and the reactivation of the AR transcriptional program (Fernandez, E. V., et al., 2015. Mol. Pharmacol. 87:1006-1012.). Resistance can be monitored by AR assessment (above) and staining for oxygenation status of the tumors with pimonidazole as described (Ragnum, H. B., et al., 2015. Br. J. Cancer 112:382-390.). Correlation of hypoxia, Ki67 (proliferation) and AR expression can help determine if resistance to the enzalutamide is arising or about to arise. Alternative investigations can be performed as described below if resistance is found.

Effect of the immune system on the treatment: Antitumor activity can be examined in the C57BL model. T- and NK-cell functionality can be assessed as reflected by enhanced cytokine secretion and cytotoxicity responses as described (Guth, A. M., Hafeman, S. D., and Dow, S. W. 2012. Depletion of phagocytic myeloid cells triggers spontaneous T cell- and NK cell-dependent antitumor activity. Oncoimmunology. 1:1248-1257).

Metabolic studies: Lipid content of the tumors can be measured by LCMS as described (Schlaepfer, I. R., et al., 2012. Mol. Cell Endocrinol. 363:111-121.). Special attention can be given to linoleic and arachidonic acid since they are associated with aggressive growth in hypoxia-reoxygenation settings (Schlaepfer, I. R., et al., 2015. Oncotarget).

ER stress in tumors: Western blot analysis of total and phosphorylated eIF2a in whole cell lysates, western blot analysis ofXBP1, ATF4, Chop, GRP78 and GADD34. All antibodies are available commercially from Cell Signaling or Santa Cruz Biotechnologies and have been validated in our laboratory. XBP1 mRNA splicing can be determined using a two-step PCR protocol and Real Time PCR can be used to monitor ATF4, Chop, GRP78, GADD34, B2 microglobulin (reference gene).

Apoptosis: Caspase-3 activity can be determined using a caspase-specific peptide conjugated to the color reporter p-nitroanaline (R&D Systems). The terminal deoxynucleotidyl transferase dUTP nick-end labeling (TUNEL) assay can also be used (Roche). Apoptosis in tumor sections can be quantified by counting the number of TUNEL-positive cells in 10 random microscopic fields (20×). Changes in expression of the apoptosis proteins like Bad, Bax and Bcl-2 can also be tested, since Bcl-2 is associated with the entry of fatty acids into the mitochondria for oxidation (Paumen, M. B et al., 1997. Biochem. Biophys. Res. Commun. 231:523-525), which is what the etomoxir drug targets.

Histology: Tumor sections can be stained for AR (Santa Cruz #N-20 & C-19) ARV-7 (Precision Antibody # AG10008), proliferation (Ki67). Two hours before sacrifice mice can be injected with Bromouridine to assess the proliferation activity in tumor sections (Schlaepfer, I. R., et al., 2012. Mol. Cell Endocrinol. 363:111-121). Pimonidazole (500 mg/m2 body surface) can be injected for assessment of hypoxia in tumors. In the TRAMPC1-C57BL/6 model, we can also stain for the presence of infiltrated T-cells in the tumors.

Secondary analysis: (1) Mouse Prostates: Mouse prostates can be examined and collected at sacrifice. They can be embedded in paraffin for histological analysis; (2) Serum Chemistry: Testosterone (EIA Sigma), Glucose (Sigma), insulin (Linco), free fatty acids (Wako), and triglycerides (Wako) can be analyzed in serum; (3) Body weight. Mouse body weight can be measured daily before treatments.

Statistics: Data can be analyzed using a 2×2 factorial design (cell line×drug) using 2-way ANOVA with Bonferonni's post hoc test. Data that fails homogeneous variance can be analyzed with the Kruskal Wallis test. The level of significance can be P<0.05. The number of animals chosen allows us to achieve a power of 80% and a minimum detectable difference of 40% for tumor growth.

Outcomes: Based on previous data (FIGS. 2 and 6) we will show that LNCaP CPT1A-KD tumors grow less than the control tumors and this decrease will be enhanced by the systemic enzalutamide treatment. These changes will be associated with increased ER stress and increased lipid accumulation (especially palmitate) in the CPT1A-KD tumors treated with enzalutamide. Regarding the TRAMC1 tumors, we expect parallel but enhanced results since the immune system might be enhanced by the dying TRAMPC1-cptlaKD cells.

Resistance to therapy: If resistance to enzalutamide arises in the control tumors (normal CPT1A expression) of the LNCaP and TRAMPC1 models, the experiment can be repeated in the presence of celecoxib, which has been shown by us to block the growth of cells in response to hypoxia-reoxygenation paradigms using the arachidonic acid accumulated during hypoxia (Schlaepfer, I. R., et al., 2015. Oncotarget, 2015 Sep. 8, 2015; 6(26):22836-56).

### Example 11—Investigation of Fat Oxidation Inhibitors in Enzalutamide-Resistant Cells In Vivo

Lipid oxidation is a characteristic of PCa cells. Systemic treatment with drugs that block fat oxidation offers promise for combinatorial therapeutic interventions. We will show that safe fat oxidation inhibitors can reduce enzalutamide-resistant (EnzaR) tumor growth by decreasing androgen action via metabolic stress. We use ranolazine because it is FDA approved and our data shows that it decreases EnzaR cell growth compared to parental cells and changes AR content. We use VCaP cells because they have a high level of AR and ARv7 expression, are sensitive to androgens and can grow well in nude mice without androgen supplementation. The TRAMPC1 cells can be used for the immune-competent mouse model.

VCaP model: Male nude mice (Charles River nu/nu) can be injected subcutaneously with 2 million VCaP-parental or VCaP-EnzaR cells suspended in Matrigel (BD Biosciences). After palpable tumors are observed, intraperitoneal drug treatment (vehicle or ranolazine: 300 mg/Kg/day) can start and last for 4 weeks. Ranolazine is partial beta-oxidation blocker that is FDA approved (FIG. 7 and (Samudio, I., et al 2010. J. Clin. Invest 120:142-156; Tocchetti, C. G., et al., 2014. Eur. J. Heart Fail. 16:358-366))

TRAMPC1 model: The TRAMPC1 model can be included to elucidate the role of fat burning inhibitors in AR action in the context of the immune system. Once tumors are established, treatment can proceed as for the VCaP model above.

Animal numbers: Each mouse can receive 2 grafts. Eight mice (16 grafts) can be used per treatment group plus 4 mice for unexpected deaths, totaling 18 mice per cell line. Thus, 36 nude and 18 C57BL/6 mice can be used (see statistical section below).

### Primary Analysis

Tumor growth and specimen collection: Tumor volumes, serum collection, and body weight can be collected as in Example 10. The VCaP models are stably transfected with a luciferase reporter construct so they can be tracked by Bioluminescence (IVIS).

Metabolic studies: Lipid metabolites, including phosphatidic acid (activator of mTOR (Foster, D. A. 2009. Biochim. Biophys. Acta 1791:949-955)) generated by the treatments can be assessed by LC/MS/MS as described (Zarini, S., Gijon, M. A., Ransome, A. E., Murphy, R. C., and Sala, A. 2009. Proc. Natl. Acad. Sci. U. S. A 106:8296-8301).

Androgen action: Indicators can be examined as in Example 10.

Effectiveness of therapy and assessment of resistance: Resistance to ranolazine therapy can be assessed as in Example 10. Effect of the immune system (C57BL/6 model only) can also be done as in Example 10.

Signaling pathways: mTOR (p-mTOR, S6K, p4EBP1) and AKT pathways can be examined since they changed with the treatments (FIG. 2).

ER Stress and Apoptosis. Markers of ER stress and apoptosis can be the same as in Example 10. Additionally, ceramide analysis can be done in tumor fragments (˜20 mg) as described (Schlaepfer, I. R., et al., 2014. Mol. Cancer Ther. 13:2361-2371; Schlaepfer, I. R., et al., 2012. Mol. Cell Endocrinol. 363:111-121.).

Histology: AR staining and makers of proliferation can be done exactly as in Example 10.

Secondary analysis: Mouse prostates, serum chemistry and body weights can be done as in Example 10.

Outcomes: Based on the data as in FIGS. 5 and 8, we will show that the enzalutamide-resistant VCaP cells will grow less with the ranolazine treatment. We expect to show that AR content (and ARv7 variant) will be decreased with drug-treatment in the enzalutamide-resistant tumors, and obliterated in the parental-derived tumors. This decrease in AR expression is should be due to unresolved lipid-mediated ER stress that blocks protein production by decreasing mTOR. In the TRAMPC1 model, we expect to show that ranolazine decreases tumor growth and blocks the immune-suppression that tumors exert over T cells, by blocking the fat oxidation of the T-cells and making them more cytolytic towards tumors.

Statistical analysis: Data can be analyzed using two-way and/or repeated measures ANOVA with Bonferonni's post hoc test. The level of significance can be P<0.05. The number of experiments can allow us to achieve a power of 80% and a minimum detectable difference of 40% for lipid and apoptotic markers.

We propose ranolazine because it is FDA approved and blocks fat oxidation in cancer cells (Samudio, I., et al., 2010. J. Clin. Invest 120:142-156), but offer etomoxir as an alternative, which is an effective CPT1 inhibitor that works well in mice.

### Example 12—Investigation of the Mechanisms Behind the Intersection of CPT1A and Androgen Action in Prostate Cancer Cell Models

Metabolic activities observed in tumor cells in vitro can be extrapolated to tumors in vivo (DeBerardinis, R. J. 2008. Genet. Med. 10:767-777), allowing the use of our primary and established cell lines from our PCa metabolic studies with confidence that the results will prove extendable. VCaP (Korenchuk, S. , Lehr, J. E., MClean, L., Lee, Y. G., Whitney, S. , Vessella, R., Lin, D. L., and Pienta, K. J. 2001. In Vivo 15:163-168). We will show that androgens promote lipid homeostasis (synthesis and catabolism via CPT1A) to promote growth (via mTOR) and avoid toxic accumulation of lipid metabolites that disrupt ER homeostasis and activate apoptotic pathways. Regarding the choice of drugs, ranolazine has been chosen because it is FDA approved and we show that it decreases EnzaR cell growth compared to parental cells and changes AR content. Etomoxir is a potent CPT1 inhibitor that is not FDA approved, although it serves as a good tool for mechanistic studies.

Cell models: Table 1, above, details the cell line models available. Additionally, 7 patient-matched pairs of primary cancer and benign cells from prostatectomies can be also used. These patient-matched pairs can provide a good control of benign prostate gland lipid metabolism.

CPT1A overexpression in LNCaP cells: LNCaP cells can be transfected with CPT1A cDNA (complete coding sequence) using a lentiviral approach (Schlaepfer, I. R., et al 2015. Mol. Imaging Biol. 17:529-538). The LNCaP KD and knockout cells can also be transfected with this CPT1A cDNA plasmid to examine the effect of CPT1A reconstitution in the cell's growth, androgen action and survival.

ER Stress mechanisms: LNCaP and VCaP cell models can be exposed to the chemical chaperones 4-phenylbyturic acid (PBA, 500 μM) or taurine-conjugated ursodeoxycholic acid (TUDCA, 200 μM) or carrier as described (Pfaffenbach, K. T., et al., 2010. Am. J. Physiol Endocrinol. Metab 298:E1027-E1035), and after 1 hr, cells can be incubated in the absence or presence of enzalutamide or ranolazine for 48 hours (n=8/group).

Mechanisms of resistance: The main problem in metastatic PCa is therapy resistance. The same approaches used for Example 10 can be used for the in vitro studies. Additionally, we can use a hypoxia-re-oxygenation paradigm to assess the role of oxygenation status in the resistance to metabolic and enzalutamide therapies. HIFl-alpha staining can be used as a marker for hypoxia.

Primary Analysis/Methods

Cell proliferation, viability and apoptosis: Cells (as described above) can be grown to 80% confluence and studied in a dose- and time-dependent manner using etomoxir, ranolazine and/or enzalutamide. MTS assays and flow-cytometry using Annexin-V can be used for viability analysis. Clonogenic assays can also be performed since they are considered long-term survival assays. Caspase-3 cleavage can used for apoptosis analysis as well. Changes in expression of the apoptosis proteins Bad, Bax and Bcl-2 can also be tested, since Bcl-2 is associated with the entry of fatty acids into the mitochondria for oxidation (Paumen, M. B., et al., 1997. Biochem. Biophys. Res. Commun. 231:523-525), which is what the etomoxir drug targets. CalcuSyn synergistic analysis (Biosoft, Ferguson, Mo.) can be used to determine the combinatorial dose that produces the best therapeutic effect.

Effectiveness of CPT1A overexpression: The increased expression of CPT1A in LNCaP cells can determined by western blot, qRTPCR and C14-Palmitate fat oxidation rate.

Androgen effects on CPT1A expression and activity: We can treat cells (parental and enzalutamide-resistant LNCaP and VCaP models) with DHT (InM) or enzalutamide (20 uM) and measure its effects on CPT1A expression by western blotting, and biochemical activity as described (Schlaepfer, I. R., et al., 2015. Mol. Imaging Biol. 17:529-538). PSA NKX3.1 and TMPRSS can be monitored as markers of androgen effects. Transfection studies with siRNA's specifically targeting the AR-full length and ARv7 and ARv567 variants can be used to assess the role of AR expression on CPT1A gene expression and activity.

CPT1A expression effects on AR expression and action: CPT1A-KD LNCaP and CPT1A overexpressing LNCaP cells can be used to study the effects of DHT (InM) or enzalutamide (20 uM) as above.

ER Stress and Apoptosis: Markers of ER stress and apoptosis, including ceramides, can be examined as discussed above in Examples 10 and 11. Since the accumulation of unfolded proteins is not only dependent on the folding but also on protein degradation, we can examine protein degradation by monitoring EDEM a protein critical to ER-associated degradation (Eriksson, K. K., et al., 2004. J. Biol. Chem. 279:44600-44605).

Signaling pathways: mTOR and AKT pathways can be examined since they changed in vitro with the treatments (prelim data). Small interfering RNA depletion of mTOR and its reconstitution (lentiviral-cDNA construct) can be used to assess mTOR involvement in the observed effects. All antibodies can be from cell signaling and they have been verified by us (Schlaepfer, I. R., et al., 2015. Mol. Imaging Biol. 17:529-538). PKA can also be examined since it is possible that BAD phosphorylation (S112) is mediated by the mTOR-PKA axis (Pringle, D. R., et al., 2014. J. Clin. Endocrinol. MAetab 99:E804-E812).

Lipid metabolism: A measure of lipid content can be as in Example 10. Special attention can be given to linoleic, arachidonic acid species, since they are associated with PCa growth and their inability to be burned with the metabolic inhibitors can make them more available for other pathways like COX2 and growth promoting eicosanoids (Schlaepfer, I. R., et al., 2015. Oncotarget).

Secondary Analysis

Gene expression analysis: LNCaP-CPT1A-KD cells and corresponding controls can be treated with enzalutamide or vehicle for 48 hours (n=3 per group) and subjected to RNAseq analysis for global gene expression at the University of Colorado array core. The data can be analyzed using Gene enrichment analysis tools. The plan for this data is to correlate it with the lipid analysis (LCMS) and biochemical studies with the goal of identifying gene signatures that reflect the effect of the treatments and the potential for resistance in the future, so additional interventions can be administered in time. For example, we can identify epigenetic modifiers (HDAC, methylases) correlated with specific lipid metabolites never explored before.

Expected outcomes: Based on data (FIGS. 4-9), we will show that decreased expression of CPT1A (shRNA KD and CRISPR-Knockout models) will result in increased palmitate accumulation, increased ER stress and increased sensitivity to apoptosis with enzalutamide treatment, mediated by compensatory increase in AR full length expression (target of enzalutamide). Treatment with etomoxir and ranolazine will parallel these effects accentuating the increased lipid-mediated ER stress and apoptosis. These effects will be reversed (decreased) in the CPT1A overexpression model. We also expect that etomoxir and ranolazine will decrease AR content and synergize with enzalutamide. Regarding the enzalutamide-resistant cells, we expect ranolazine will effectively reduce AR full length and variants leading to enhanced sensitivity to enzalutamide.

Statistical analysis: Cell culture experiments can be analyzed by two-way ANOVA with Bonferonni's post hoc test. Data that fails Bartlett's test for homogeneous variance can be analyzed with the Kruskal Wallis test. The level of significance can be P<0.05. The number of experiments chosen for in vitro studies allows us to achieve a power of 80% and a minimum detectable difference of 30% for pathway markers.

Alternative Studies:

Focus on enzalutamide: Proposed experiments are focused on enzalutamide because it is used currently in the clinic and our data shows synergy with lipid catabolism via CPT1A. However, we would also test Galeterone (AR and CYPI 7 inhibitor, phase 2 trials) and AR-509 (AR inhibitor, phase 3 trials). Galeterone appears to target the AR full length receptor as well as the variants with truncated c-terminus, like ARv7. Thus, the effect of ranolazine could be more potent since it appears to upregualte ARv7 in VCaP cells, FIG. 8C. Both anti-androgen drugs are available from Sellekchem.com.

### Glossary of Claim Terms

### Definitions

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” whereever used herein includes the meaning of “and”, “or” and “all or any other combination of the elements connected by said term”.

The term “about” or “approximately” as used herein means within 20%/o, preferably within 10%, and more preferably within 5% of a given value or range.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

The term “administration” and variants thereof (e.g., “administering” a compound) in reference to a compound of the invention means introducing the compound into the system of the subject in need of treatment. When a compound of the invention is provided in combination with one or more other active agents (e.g., a cytotoxic agent, etc.), “administration” and its variants are each understood to include concurrent and sequential introduction of the compound and other agents.

As used herein, the term “administering” includes activities associated with providing a patient an amount of a compound described herein, e.g., a drug that blocks or reduces lipid metabolism such as ranolazine, perhexiline, and/or etomoxir. Administering includes providing unit dosages of compositions set forth herein to a patient in need thereof. Administering includes providing effective amounts of compounds, e.g., a pharmaceutical composition that blocks or reduces lipid metabolism (e.g., ranolazine, perhexiline, etomoxir), for specified period of time, e.g., for about 1, 2, 3, 4, 5, 6, 7, 8, 9, 10, 12, 13, 14, 15, 16, 17, 18, 19, 20, 21, 22, 23, 24, 25, 26, 27, 28, 29, 30, or 31 days, or in a specified sequence, e.g., administration of a pharmaceutical composition that blocks or reduces lipid metabolism (e.g., ranolazine, perhexiline, etomoxir) followed by the administration of a compound selected from the group consisting of enzalutamide, abiraterone, docetaxel, bicalutamide, and combinations thereof, or vice versa.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation or metastasis of the tumor. In some embodiments, an effective amount is an amount sufficient to delay development. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis: (v) inhibit tumor growth; (vi) prevent or delay occurrence and/or recurrence of tumor; and/or (vii) relieve to some extent one or more of the symptoms associated with the cancer.

An effective amount of a pharmaceutical composition that blocks or reduces lipid metabolism (e.g., ranolazine, perhexiline, etomoxir) includes an amount sufficient to alleviate the signs, symptoms, or causes of prostate cancer. Thus, an effective amount can be an amount that slows or reverses tumor growth, increases mean time of survival, inhibits tumor progression or metastasis, or resensitizes a prostate cancer cell to a prostate cancer drug to which it has become or is resistant. Also, for example, an effective amount of a pharmaceutical composition that blocks or reduces lipid metabolism (e.g., ranolazine, perhexiline, etomoxir) includes an amount sufficient to cause a substantial improvement in a subject having prostate cancer when administered to the subject. The amount may vary with the type of prostate cancer being treated, the stage of advancement of the prostate cancer, the type and concentration of composition applied, and the amounts of anti-androgens drugs that are also administered to the subject. For example, an effective amount of a pharmaceutical composition that blocks or reduces lipid metabolism (e.g., ranolazine, perhexiline, etomoxir) can include an amount that is effective in enhancing the prostate cancer therapeutic activity of drugs such as enzalutamide, abiraterone, docetaxel, and bicalutamide.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, preventing or delaying spread (e.g., metastasis) of the cancer, preventing or delaying occurrence or recurrence of cancer, delay or slowing of cancer progression, amelioration of the cancer state, and remission (whether partial or total). The methods of the invention contemplate any one or more of these aspects of treatment.

As used herein, the term “treating” includes, but is not limited to, methods and manipulations to produce beneficial changes in a recipient's health status, e.g., a patient's prostate cancer status. The changes can be either subjective or objective and can relate to features such as symptoms or signs of the prostate cancer being treated. For example, if the clinician notes objective changes, such as reducing the number of prostate cancer cells, the growth of the prostate cancer cells, the size of prostate cancer tumors, or the resistance of the prostate cancer cells to another prostate cancer drug, then treatment of prostate cancer has been beneficial. Preventing the deterioration of a recipient's status is also included by the term. Treating, as used herein, also includes administering a pharmaceutical composition that blocks or reduces lipid metabolism (e.g., ranolazine, perhexiline, etomoxir) to a patient having prostate cancer.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g., sequentially or simultaneously, via intravenous or intraperitoneal injection.

As used herein, the phrase “advanced stage prostate cancer” or “advanced prostate cancer” includes a class of prostate cancers that has progressed beyond early stages of the disease. Typically, advanced stage prostate cancers are associated with a poor prognosis. Types of advanced stage prostate cancers include, but are not limited to, metastatic prostate cancer, drug-resistant prostate cancer such as anti-androgen-resistant prostate cancer (e.g., enzalutamide-resistant prostate cancer, abiraterone-resistant prostate cancer, bicalutamide-resistant prostate cancer, and the like), hormone refractory prostate cancer, castration-resistant prostate cancer, metastatic castration-resistant prostate cancer, and combinations thereof. In some instances, the advanced stage prostate cancers do not generally respond, or are resistant, to treatment with one or more of the following conventional prostate cancer therapies: enzalutamide, arbiraterone, bicalutamide, and docetaxel. Compounds, compositions, and methods of the present invention are provided for treating prostate cancer, such as advanced stage prostate cancer, of the types of advanced stage prostate cancers disclosed herein.

As used herein, the phrase “ameliorating the symptoms of prostate cancer’ includes aleviating or improving the symptoms or condition of a patient having prostate cancer. Ameliorating the symptoms includes reducing the pain or discomfort associated with prostate cancer. Amerliorating the symptoms also includes reducing the markers of prostate cancer, e.g., reducing the number of prostate cancer cells or reducing the size of prostate cancer tumors.

As used herein, the phrase “enhancing the therapeutic effects” includes any of a number of subjective or objective factors indicating a beneficial response or improvement of the condition being treated as discussed herein. For example, enhancing the therapeutic effects of an anti-androgen drug (e.g., enzalutamide, abiraterone, or bicalutamide) includes resensitizing anti-androgen drug resistant prostate cancer to anti-androgen therapy. Also, for example, enhancing the therapeutic effects of an anti-androgen drug includes altering anti-androgen drug resistant prostate cancer cells so that the cells are not resistant to anti-androgen drugs. Also, for example, enhancing the therapeutic effects of an anti-androgen drug includes additively or synergistically improving or increasing the activity of the anti-androgen drug.

As used herein, the phrase “reversing prostate cancer cell resistance” includes altering or modifying a prostate cancer cell that is resistant to anti-androgen drug therapy so that the cell is no longer resistant to anti-androgen drug therapy.

As used herein, the phrase “reducing prostate cancer cell resistance” includes increasing the therapeutic activity of an anti-androgen drug towards prostate cancer cells that are, or previously were, resistant to anti-androgen drug therapy.

As used herein, the phrase “resensitizing prostate cancer cell resistance” includes inducing sensitization towards anti-androgen drug therapy in prostate cancer cells which are resistant to anti-androgen drug therapy. Sensitization as used herein includes inducing the ability of a prostate cancer cell to be effectively treated with anti-androgen drugs. Sensitization also includes reducing the dosage required to achieve a beneficial effect with anti-androgen drug therapy.

As used herein, the phrase “anti-androgen drug” includes anti-androgen compounds that alter the androgen pathway by blocking the androgen receptors, competing for binding sites on the cell's surface, or affecting or mediating androgen production. Anti-androgens are useful for treating several diseases including, but not limited to, prostate cancer. Anti-androgens include, but are not limited to, enzalutamide, abiraterone, and bicalutamide.

As used herein, the term “androgen receptor” or “AR” includes a nuclear receptor that binds androgenic hormones testosterone or dihydrotestosterone in the cytoplasm and translocates to the nucleus. AR modulates, inter alia, transcription of target genes by binding to Androgen Response Elements (AREs) in the promoters of such target genes.

The term “subject,” “individual,” or “patient” typically includes humans, but can also include other animals such as, e.g., other primates, rodents, canines, felines, equines, and the like.

A “subject in need of treatment” is a mammal with cancer that is life-threatening or that impairs health or shortens the lifespan of the mammal.

A “pharmaceutically acceptable” or “therapeutically acceptable” component is one that is suitable for use with humans and/or animals without undue adverse side effects (such as toxicity, irritation, and allergic response) commensurate with a reasonable benefit/risk ratio and which does not interfere with the effectiveness or the biological activity of the active ingredients.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

Other than in the operating examples, or unless otherwise expressly specified, all of the numerical ranges, amounts, values and percentages such as those for amounts of materials, times and temperatures of reaction, ratios of amounts, values for molecular weight (whether number average molecular weight (“Mₙ”) or weight average molecular weight (“M_{w}”), and others in the following portion of the specification may be read as if prefaced by the word “about” even though the term “about” may not expressly appear with the value, amount or range. Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used.

As used herein, the term “pretreating”, or “pretreatment”, is intended to mean that a first treatment is administered prior to, or in conjunction with, a second treatment. In other words, the pretreatment may be performed before another, later treatment, thus allowing the pretreatment time to take effect. Alternatively, the pretreatment may be performed or administered simultaneously with a second treatment without a temporal delay. Advantageously, a pretreatment is administered prior to a second treatment. It is envisioned that pretreatment with a chemotherapeutic agent can be performed 1 hr., 2 hrs., 4 hrs., 8 hrs., 1 day, 2 days, 4 days, 1 week, 2 weeks, or 1 month prior to treatment.

The present invention also provides a method for treating a patient, comprising administering to said patient simultaneously or sequentially a therapeutically effective amount of a combination of the anti-cancer agent. In one embodiment the patient is a human that is being treated for cancer. In different embodiments, the anti-cancer agent or treatment are co-administered to the patient in the same formulation; are co-administered to the patient in different formulations; are co-administered to the patient by the same route; or are co-administered to the patient by different routes. In another embodiment one or more other anti-cancer agents can additionally be administered to said patient with the anti-cancer agent/treatment combination. Furthermore, for any of the methods, compositions or kits of the invention described herein, this invention also includes a corresponding method, composition or kit.

Kits for practicing the methods of the invention are further provided. By “kit” is intended any manufacture (e.g., a package or a container) comprising at least one reagent, e.g., a pH buffer of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

In an advantageous embodiment, the kit containers may further include a pharmaceutically acceptable carrier. The kit may further include a sterile diluent, which is preferably stored in a separate additional container. In another embodiment, the kit further comprising a package insert comprising printed instructions directing the use of a combined treatment of an pH buffer and the anti-cancer agent as a method for treating tumors, tumor metastases, or other cancers in a patient. The kit may also comprise additional containers comprising additional anti-cancer agents, agents that enhances the effect of such agents, or other compounds that improve the efficacy or tolerability of the treatment.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

The anti-cancer agent or treatment will typically be administered to the patient in a dose regimen that provides for the most effective treatment of the cancer (from both efficacy and safety perspectives) for which the patient is being treated, as known in the art. In conducting the treatment method of the present invention, the anti-cancer agent or treatment can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of anti-cancer agent or treatment being used, and the medical judgment of the prescribing physician as based, e.g., on the results of published clinical studies.

The anti-cancer agent or treatment can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

Methods of preparing pharmaceutical compositions comprising anti-cancer agents or treatments are known in the art. Methods of preparing pharmaceutical compositions are also known in the art. In view of the teaching of the present invention, methods of preparing pharmaceutical compositions comprising an anti-cancer agent or treatment will be apparent from the art, from other known standard references, such as Remington's Pharmaceutical Sciences, Mack Publishing Company, Easton, Pa., 18^{th} edition (1990).

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.).

The receding examples are provided for the purpose of illustration and are not intended to limit the scope of the present invention.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of treating prostate cancer in a subject comprising the step of:
administering to the subject a pharmaceutically effective amount of enzalutamide in combination with a pharmaceutically effective amount of ranolazine or etomoxir.

2. The method according to claim 1 where the subject being treated for cancer has prostate cancer that is unresponsive or has a reduced responsiveness to anti-androgen treatment.

3. The method of treating cancer in a subject according to claim 1 wherein the subject is pretreated with ranolazine or etomoxir and the subject is treated with enzalutamide.

4. The method according to claim 3 wherein the cancer is prostate cancer.

5. The method according to claim 4 where the prostate cancer is unresponsive or has a reduced responsiveness to anti-androgen treatment.

6. The method according to claim 3 wherein the pretreatment is performed about 1 hr., about 2 hrs., about 4 hrs., about 8 hrs., about 1 day, about 2 days, about 4 days, about 1 week, about 2 weeks, or about 1 month prior to treatment.

7. The method according to claim 1 further comprising the step of administering celecoxib to the subject.

8. A combination therapy for the treatment of treatment of prostate cancer comprising:
a pharmaceutically effective amount of enzalutamide, abiraterone, bicalutamide, galeterone, or AR-509; and
a pharmaceutically effective amount of ranolazine, perhexiline, or etomoxir.

9. The combination therapy according to claim 8 further comprising a pharmaceutically effective amount of celecoxib.

10. A method of treating cancer in a subject comprising the steps of:
administering a first drug to the subject wherein the first drug is enzalutamide, abiraterone, bicalutamide, galeterone, or AR-509;
assessing the responsiveness of the cancer in the subject to the administration of the first drug; and
administering a second drug to the subject responsive to the assessment that the cancer of the subject has limited or no response to the first drug wherein the second drug is ranolazine, perhexiline, or etomoxir.

11. The method according to claim 10 wherein the cancer is prostate cancer.

12. The method according to claim 6 wherein enzalutamide, abiraterone, bicalutamide, galeterone, or AR-509 is administered in combination with the administration of the second drug following the assessing step.

13. The method according to claim 12 further comprising the step of administering celecoxib to the subject in combination with the second drug.

14. The method according to claim 10 further comprising the step of administering celecoxib to the subject in combination with the second drug.
